(19) United States
(12) Patent Application Publication
(10) Pub. No.: US 2025/0092089 A1
(43) Pub. Date: Mar. 20, 2025

(54) METHODS FOR PURIFYING TARGET BIOLOGICS

(71) Applicants: North Carolina State University, Raleigh, NC (US); Merck Sharp and Dohme LLC, Rahway, NJ (US); EMD Millipore Corporation, Burlington, MA (US); Rensselaer Polytechnic Institute, Troy, NY (US)

(72) Inventors: Stefano Menegatti, Raleigh, NC (US); Ruben G. Carbonell, Raleigh, NC (US); Jacob P. LeBarre, Raleigh, NC (US); Cristiana Boi, Raleigh, NC (US); Steven Cramer, Niskayuna, NY (US); Michael Phillips, Burlington, MA (US); David Roush, Rahway, NJ (US)

(21) Appl. No.: 18/890,289

(22) Filed: Sep. 19, 2024

Related U.S. Application Data
(60) Provisional application No. 63/583,755, filed on Sep. 19, 2023.

(51) Int. Cl. C07K 1/16; B01D 15/34; B01D 15/36

(52) U.S. Cl. CPC C07K 1/165; B01D 15/34; B01D 15/361

(57) ABSTRACT

The present disclosure provides methods related to the purification of a biologic. In particular, the present disclosure methods for purifying a biologic using a multifunctional chromatography medium comprising porous beads or particles having a defined pore diameter distribution and one or more ligands conjugated to the surface of the pores capable of removing process-related impurities and product-related impurities from a target biologic.

### CROSS REFERENCE TO RELATED APPLICATIONS

This application claims priority to and the benefit of U.S. Provisional Patent Application No. 63/583,755 filed Sep. 19, 2023, which is incorporated herein by reference in its entirety for all purposes.

### GOVERNMENT SUPPORT

This invention was made with government support under grant number 70NANB17H002 awarded by the National Institute of Standards & Technology. The government has certain rights in the invention.

### FIELD

The present disclosure provides materials and methods related to the purification of a biologic. In particular, the present disclosure provides a multifunctional chromatography medium comprising porous beads or particles, or a monolith having a defined pore diameter distribution and one or more ligands conjugated to the surface of the pores capable of capturing process-related impurities and product-related impurities from a target biologic.

### BACKGROUND

The recent advent of diverse formats of protein therapeutics derived from monoclonal antibodies (mAbs), e.g., crossmAb, IgG-scFv, IgG-dsFv, DVD-IgG, and the like, show great promise to improve the outcome of immunotherapy. Their complex architecture, however, imposes a significant stress on the expression hosts, resulting in increased titers of process-related (host cell proteins and DNA) and product-related (aggregates and fragments) impurities. Antibody fragments can result from incorrect overexpression of portions of the construct, improper annealing of heavy and light chains, or chemical and enzymatic degradation of the product. Antibody fragments, whether single domains or “clipped” forms, may possess limited or no therapeutic activity, and their clearance, prescribed by bioprocess regulations, often represents a significant burden on downstream processing.

Current purification processes rely on ion-exchange and hydrophobic interaction chromatography, or their combination in mixed-mode (MM) formats, to isolate the product of interest from product-related impurities by leveraging often subtle differences in charge or amphiphilicity. To date, a limited body of work has been dedicated to studying the contribution of different antibody domains to chromatographic binding or the removal of antibody fragments and aggregates in bind-and-elute mode. Furthermore, most of this literature focuses on defined Fab and Fc species, while in reality the biomolecular landscape of fragments is significantly more diverse. The application of flow-through MM chromatography to the removal of mAb fragments and clipped forms is even more limited, despite its ability to enable continuous processes and higher throughput as well as lower capital and operational costs. The lack of chromatographic technology enabling robust clearance of antibody fragments represents a significant gap in the state of the art that is yet to be properly addressed.

Thus, there is a need for tools which enable a flexible platform for purification of any biologic, not limited to antibodies, which comprises steps operated in flow-through mode to remove process- and product-related impurities while allowing the desired product to flow-through un-bound.

### SUMMARY

Embodiments of the present disclosure include methods for purifying a target biologic. The methods comprise contacting a sample comprising the target biologic with a multifunctional chromatography medium; and collecting the target biologic. In some embodiments, the multifunctional chromatography medium comprises a plurality of beads or particles having pores, wherein the plurality of beads or particles has a defined pore diameter distribution, and one or more ligands functionalized to surfaces of the pores. In some embodiments, the one or more ligands are configured to bind or associate with at least one impurity in the sample.

In some embodiments, the target biologic is one or more of a protein, peptide or polypeptide; an oligonucleotide or a polynucleotide; a virus or a virus-like particle; an exosome or an extracellular vesicle; a cell or cell organelle; or a small molecule. In some embodiments, the target biologic is an antibody.

In some embodiments, the at least one impurity comprises a process-related impurity, a product-related impurity, or a combination thereof. In some embodiments, the at least one impurity comprises a fragment of the target biologic, and the method comprises separating the target biologic from the fragment of the target biologic.

In some embodiments, the ligand has a high binding strength for the at least one impurity. In some embodiments, the ligand binds or associates to the at least one impurity with a dissociation constant (K_{d}) of about 10⁻³ M to about 10⁻⁹ M. In some embodiments, the at least one impurity is part of an aggregate comprising the target biologic and the at least one impurity is partitioned from the aggregate.

In some embodiments, the plurality of beads or particles has a narrow pore diameter distribution. In some embodiments, the pore diameter distribution is configured to exclude a target biologic. In some embodiments, the pore diameter distribution has an average pore diameter of about 50 Å to about 1000 Å. In some embodiments, the average pore diameter is about 100 Å to about 500 Å. In some embodiments, the pore diameter distribution is monodisperse.

In some embodiments, plurality of beads or particles may be silica, polyacrylamide, polymethacrylate, polystyrene, polyvinylether, or combinations thereof. In some embodiments, the plurality of beads or particles are silica. In some embodiments, the plurality of beads or particles have a particle size ranging between 20 to 150 μm.

In some embodiments, the one or more ligands are: a small molecule, a biomolecule, a metal, or a combination thereof. In some embodiments, the biomolecule comprises a nucleic acid, an oligonucleotide, an amino acid, a peptide, a monosaccharide, an oligosaccharide, a lipid, a steroid, or a conjugate thereof. In some embodiments, each of the one or more ligands comprise an affinity ligand, an ion-exchange ligand, a hydrophobic ligand, a hydrophilic ligand, a polar ligand, a thiophilic ligand, or a combination thereof.

In some embodiments, the ion exchange ligand is negatively charged. In some embodiments, the ion exchange ligand comprises a sulfate group, a phosphate group, a carboxylate group, or a combination thereof. In some embodiments, the ion exchange ligand is positively charged. In some embodiments, the ion exchange ligand comprises a primary, secondary, tertiary, or quaternary aminoalkyl group, an ammonium group, or an oligomer thereof or a polymer thereof; an imidazole group; a pyridinyl group; a guanidyl group; or a combination thereof.

In some embodiments, the hydrophobic ligand comprises alkyl or aryl groups.

In some embodiments, the affinity ligand is a biological affinity ligand, an immunoaffinity ligand, or a metal affinity ligand.

In some embodiments, the chromatography medium is suspended in solution or packed in a column. In some embodiments, the method is performed under static binding conditions. In some embodiments, the method comprises collecting the target biologic in the unbound fraction, wash fraction, or a combination thereof. In some embodiments, the method is performed under dynamic binding conditions. In some embodiments, the method comprises collecting the target biologic in the flow through, column wash, or a combination thereof.

In some embodiments, the methods further comprise preparing the sample comprising the target biologic. In some embodiments, preparing the sample comprising the target biologic comprises one or more chromatography steps. In some embodiments, preparing the sample comprises carrying out affinity chromatography and collecting an eluate comprising the target biologic.

In some embodiments, the methods further comprise one or more additional chromatography steps following collecting the target biologic.

In some embodiments, the methods further comprise selecting the multifunctional chromatography medium.

Other aspects and embodiments of the disclosure will be apparent in light of the following detailed description and accompanying figures.

### BRIEF DESCRIPTION OF THE DRAWINGS

FIG. 1: Operating principle of SEMM-Silica resin continuously loaded with a feed stream containing intact and fragmented antibodies: the fragments access the pores of the Chromatorex silica resin, where they are captured by the MM ligands, while the full antibody flows through unbound.

FIGS. 2A-2G: Structure of the mixed-mode ligands introduced in this study: (A) APTES, (B) TMTAC, (C) DTS, (D) SBA-APTES, (E) SBA-APTES/TMTAC, (F) CA-1-Na1-APTES, and (G) SBA-Lys(Me)3-APTES.

FIG. 3: Isotherm binding curves constructed by incubating solutions of human IgG with titer ranging from 1 to 40 g/L in either 50 mM Phosphate buffer at pH 7, or 50 mM Tris HCl buffer at pH 8 with APTES-Chromatorex, TMTAC-Chromatorex, and DTS-Chromatorex resin with pore size of 300 Å, 200 Å, or 100 Å. The values of maximum binding capacity (Qₘₐₓ) and strength (i.e., dissociation constant, K_{D}) are listed in Table 1.

FIG. 4: Isotherm binding curves constructed by incubating solutions of Fab and Fc fragments of human IgG in either 50 mM Phosphate buffer at pH 7 or 50 mM Tris HCl buffer at pH 8 with concentration varying between 0.25 and 2 g/L with APTES-Chromatorex, TMTAC-Chromatorex, and DTS-Chromatorex resins with pore size of 100 Å. The values of maximum binding capacity (Qₘₐₓ) and strength (i.e., dissociation constant, K_{D}) are listed in Table 8.

FIG. 5: Simulated breakthrough curves of monomer IgG (red), Fab₂ (cyan), Fab/Fc (pink), and LMW (blue) produced by model resins with pore diameter of 100, 200, and 300 Å operated at the residence time (RT) of 2, 6, and 10 min. The concentration of each species is normalized and reported as C/Co over the 15 CVs load and 2 CVs wash.

FIGS. 6A-6C: Values of cumulative monomeric IgG purity calculated with respect to the capture of (A) IgG fragments only (CP_{F}) and (B) IgG fragments and aggregates (CP_{A+F}), and (C) cumulative yield of monomeric IgG (CY %) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in 50 mM sodium phosphate buffer at pH 7 on APTES-Chromatorex resin at the linear velocity of either 200, 150, 120, 100, or 85.7 cm/h, corresponding to residence times of 3, 4, 5, 6, and 7 min. The corresponding values of fractional monomeric IgG purity and yield and presented in FIG. 10.

FIGS. 7A-7C: Values of cumulative monomeric IgG purity calculated with respect to the capture of IgG fragments only (P_{C,F}) or IgG fragments and aggregates (P_{C,A+F}), and cumulative yield of monomeric IgG (Y_{C}%) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in either 50 mM sodium phosphate buffer at pH 6 or 7, or 50 mM Tris HCl at pH 8 and 9 on (A) APTES-Chromatorex, (B) TMTMAC-Chromatorex, and (C) DTS-Chromatorex resin at the linear velocity of 100 cm/h, corresponding to a residence time of 6 min. The corresponding values of fractional monomeric IgG purity and yield and presented in FIG. 11.

FIGS. 8A-8C: Values of cumulative monomeric IgG purity calculated with respect to the capture of (A) IgG fragments only (P_{C,F}) and (B) IgG fragments and aggregates (P_{C,A+F}), and (C) cumulative yield of monomeric IgG (Y_{C}%) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in 50 mM sodium phosphate buffer and X mM NaCl (X: 0, 25, 50, and 150) at pH 7 on DTS-Chromatorex resin at the linear velocity of 100 cm/h, corresponding to a residence time of 6 min. The corresponding values of fractional monomeric IgG purity and yield and presented in FIG. 12.

FIGS. 9A-9H: Chromatograms generated by loading a solution of human either IgG (15 mg/mL), or Fc fragment (0.25 mg/mL) or Fab fragment (0.25 mg/mL) prepared in 50 mM Tris HCl buffer at pH 8.0 onto a 0.3 mL column loaded with (A) no resin, (B) DTS-Chromatorex resin, (C) TMTMAC-Chromatorex resin, (D) SBA-Chromatorex resin, (E) SBA/TMTMAC-Chromatorex resin, (F) SBA-Lys(Me)₃-Chromatorex resin, (G) CA-1-Na1-Chromatorex resin, and (H) APTES-Chromatorex resin (pore diameter: 100 Å) at 173 cm/h while continuously monitoring the UV absorbance of the effluent via UV spectroscopy at 280 nm.

FIGS. 10A-10C: Values of fractional monomeric IgG purity calculated with respect to the capture of (A) IgG fragments only (P_{F,F}) and (B) IgG fragments and aggregates (P_{F,A+F}), and (C) fractional yield of monomeric IgG (Y_{F}%) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in 50 mM sodium phosphate buffer at pH 7 on APTES-Chromatorex resin at the linear velocity of either 200, 150, 120, 100, or 85.7 cm/h, corresponding to residence times of 3, 4, 5, 6, and 7 min.

FIGS. 11A-11C: Values of fractional monomeric IgG purity calculated with respect to the capture of IgG fragments only (P_{F,F}) or IgG fragments and aggregates (P_{F,A+F}), and fractional yield of monomeric IgG (Y_{F}%) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in either 50 mM sodium phosphate buffer at pH 6 or 7, or 50 mM Tris HCl at pH 8 and 9 on (A) APTES-Chromatorex, (B) TMTMAC-Chromatorex, and (C) DTS-Chromatorex resin at the linear velocity of 100 cm/h, corresponding to a residence time of 6 min.

FIGS. 12A-12C: Values of cumulative monomeric IgG purity calculated with respect to the capture of (A) IgG fragments only (P_{F,F}) and (B) IgG fragments and aggregates (P_{F,A+F}), and (C) cumulative yield of monomeric IgG (Y_{F}%) obtained by loading a model Protein A eluate containing human monomeric IgG (5 g/L), IgG aggregate (0.35 g/L), Fab₂ (0.025 g/L), Fc/Fab (0.28 g/L), and low molecular weight fragments (0.02 g/L) in 50 mM sodium phosphate buffer and X mM NaCl (X: 0, 25, 50, and 150) at pH 7 on DTS-Chromatorex resin at the linear velocity of 100 cm/h, corresponding to a residence time of 6 min.

FIG. 13: Breakthrough curves obtained by loading human monomeric IgG at 5 mg/mL (grey), and F(ab)₂ (blue), Fab (orange), and Fc (green) fragments at 2 mg/mL in 50 mM sodium phosphate buffer and 25 mM NaCl at pH 7 on DTS-Chromatorex resin at the linear velocity of 100 cm/h, corresponding to a residence time of 6 min.

### DETAILED DESCRIPTION

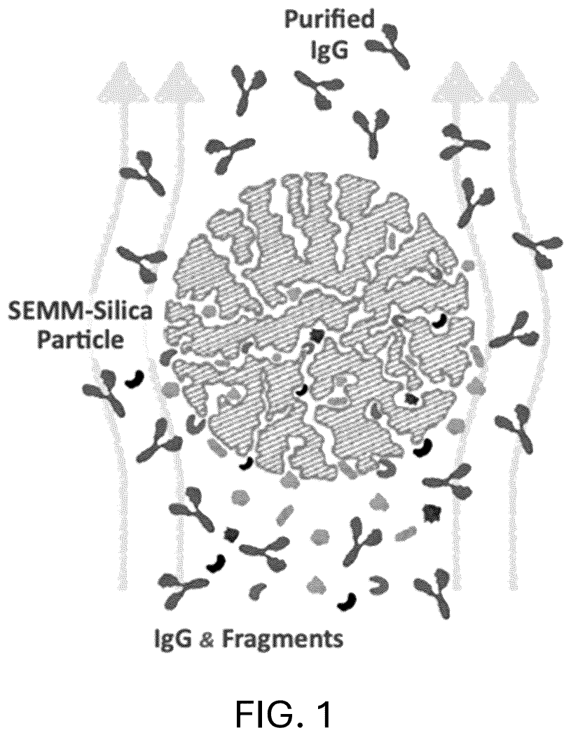

Embodiments of the present disclosure establish a chromatography medium and related methods for use in purifying biologics which efficiently and effectively removes contaminants from the sample or solution comprising the biologic. In accordance with these embodiments, the disclosed chromatography medium combines the size-exclusion capability of silica beads with the broad biorecognition activity of various ligands (FIG. 1). The diameter of the pores in the silica resin is highly monodisperse and can be selected to exclude the target biologic while facilitating access to impurities. For example, the size-exclusion resin can be selected to exclude monomeric antibodies while allowing access to its subdomains (Fc, Fab, and F(ab)₂) and clipped forms. The pore surface of the size-exclusion resin is functionalized with ligands, e.g., ligands that feature strong ion-exchange and hydrophobic moieties, thus ensuring high binding capacity and strong retention of the impurities. Additionally, the rigidity of the silica particles minimizes the risk of bead compaction when operating within a broad range of flowrates and numerous purification cycles. The combination of high capacity and flow-through operation provides for a substantial reduction of process footprint and capital costs, while the low cost of both resin and ligands enables single-use, thus removing the burden of cleaning and validation, ultimately operational costs. Furthermore, by operating in flow-through mode, the disclosed chromatography media are conducive to continuous purification processing. Most flow-through chromatography to date has focused on the capture of process-related impurities, chiefly host cell proteins and DNA, whereas the disclosed chromatography medium is capable of removing both process- and product-related impurities in a single flow-through chromatography process.

As shown herein, an exemplary chromatography medium had a static and dynamic binding capacity between 30-45 and 25-33 grams of antibody fragments per mL of resin, respectively. Selected chromatography media also demonstrated the ability to capture antibody aggregates, which adsorb on the outer layer of the beads. Optimization of the chromatography media and operation conditions, for example pore size, ligand composition, linear velocity, ionic strength, and pH of the mobile phase, afforded a significant reduction of both fragments and aggregates, resulting in a final antibody yield up to 80% and monomeric purity above 97%.

Collectively, the disclosed chromatography media is a viable tool for next-generation manufacturing processes of biologics.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

As used herein, the term “about” refers to plus or minus up to 10% of the value it modifies (rounded up to the nearest whole number if the value is not sub-dividable, such as a number of molecules or nucleotides). The term “about”, when modifying the quantity (e.g., mg) of a substance or composition, a parameter of a substance or composition or a parameter used in characterizing a step in a method, or the like, refers to variation in the numerical quantity that can occur. Such variation can occur: through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein means compared to.

The term “pore diameter distribution” refers to a probability density function that specifies what fraction of the pore volume has a specific value of pore diameter. A pore diameter distribution provides a spectrum of porosity based on the relative abundance of pores in a given chromatography material, such as a particle or bead. The pore radius distribution is defined by the equation:

$$p ( r ) = \frac{1}{{V}_{P}} \frac{dV}{dr}$$

Wherein r is the pore radius (½ of the pore diameter), V is the volume of pores having radius r, and V_{P} is the total pore volume; r varies from a minimum value rₘᵢₙ to a maximum value rₘₐₓ. Since:

$${\int }_{{r}_{min}}^{{r}_{max}} p ( r ) dr = {\int }_{{r}_{min}}^{{r}_{max}} \frac{1}{{V}_{P}} \frac{dV}{dr} dr = \frac{1}{{V}_{P}} {\int }_{{r}_{min}}^{{r}_{max}} dV = \frac{{V}_{P}}{{V}_{P}} = 1$$

The term “average pore radius” (?) is defined by the equation:

$${\int }_{{r}_{min}}^{\overline{r}} p ( r ) dr = {\int }_{\overline{r}}^{{r}_{max}} \frac{1}{{V}_{P}} \frac{dV}{dr} dr = \frac{1}{2}$$

Namely, r is the value of pore radius which defines 50% of the cumulative pore size distribution

$$( \int \frac{1}{{V}_{p}} \frac{dV}{dr} dr ) .$$

Accordingly, a “narrow pore size distribution” is defined as that where the cumulative pore size encompassed by values of the radius immediately surrounding the average pore radius (r̅) approximates the total pore volume, as defined by the equation:

$${\int }_{{\overline{r}}^{-}}^{{\overline{r}}^{+}} p ( r ) dr \sim {V}_{p}$$

Wherein r̅⁻ and r̅⁺ represent values of pore radius that are respectively immediately below and above the average pore radius. The narrower the pore size distribution, the more r̅⁻˜r̅⁺.

Quantitatively, the values of r̅⁻ and r̅⁺ can be represented respectively as decrements and increments of r̅, for example: r̅⁻=0.95 r̅ and r̅⁺=1.05 r̅; or r̅⁻=0.9 r̅ and r̅⁺=1.1 r̅; and the like. Accordingly,

$${\int }_{{\overline{r}}^{-}}^{{\overline{r}}^{+}} p ( r ) dr = 0.9 {V}_{p} or 0.95 {V}_{P}$$

The term “chromatography,” as used herein, refers to a technique which separates the product of interest (e.g., a therapeutic protein or antibody) from contaminants and/or protein aggregates in a biopharmaceutical preparation.

The terms “flow-through process,” “flow-through mode,” and “flow-through chromatography,” as used interchangeably herein, refer to a product separation technique in which a sample containing the product of interest is intended to flow-through a material (e.g., a chromatography medium). In some embodiments, the product of interest flows through the material and various impurities bind to the material.

As used herein, the terms “purified” and “purification” refer to the removal of contaminants or impurities from a sample, for example, the removal of process-related impurities (e.g., soluble molecules that are not the product or derived from the product) and/or product-related impurities (e.g., soluble molecules that are derived from the product but they are not identical to the product in terms of amino acid sequence, tertiary/quaternary structure, post-translational modifications, and functional activity). In some aspects, antibodies are purified by removing various impurities such as non-immunoglobulin proteins, DNA and RNA, soluble components generated/released by cells, and various other soluble components present in the cell culture media. Antibodies are also purified by removing protein fragments and/or aggregates of the antibody. The removal of non-immunoglobulin proteins and/or the removal of immunoglobulin fragments or aggregates results in an increase in the percent of target immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” refers to any composition or mixture that contains a target biologic. Samples may be derived from biological or other sources. Biological sources include prokaryotic sources and eukaryotic sources, the latter including fungal, vegetal, and animal cells, tissues, and organs. The sample may also include diluents, buffers, additives, and contaminating species, debris and the like that are found mixed with the target biologic. The sample may be “partially purified” (e.g., having been subjected to one or more purification steps, such as filtration steps) or may be obtained directly from a host cell or organism producing the target molecule (e.g., the sample may comprise harvested cell culture fluid).

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an antibody or any antigen binding fragment/derivative thereof (e.g., monoclonal or polyclonal antibody). In other embodiments, the target or target biologic is a viral vector (e.g., AAV).

As used herein, the term “host cell protein” or “HCP” refers to any intracellular or secreted protein produced by the organism engineered to produce the target biologic and unrelated to the target biologic. HCPs are generally undesirable in the final drug substance.

As used herein, the term “ligandability” describes the propensity of a biomolecule called the “ligate” to bind molecule called the “ligand” with high affinity (e.g., the dissociation constant, K_{d}, between the ligand and the biomolecule is less than about 10⁻³ M). In some embodiments, the biomolecule is an impurity or contaminant. In some embodiments, the ligand is conjugated to a chromatographic medium.

### 2. Chromatography Media

The present disclosure provides chromatography media, e.g., for purifying a target biologic from one or more product- and/or process-related impurities or contaminants. In some embodiments, the media, and related methods, disclosed herein facilitate the flow-through purification and isolation of a target biologic from the one or more product- and/or process-related impurities or contaminants.

The disclosed chromatography medium is multifunctional in that it separates the target biologic by leveraging two orthogonal separation methods, namely, (a) the size difference between a target biologic and impurities, and (b) the ligandability of impurities. To facilitate (a), the chromatography medium includes pores that allow impurities to access the interior of the chromatography medium while excluding the target biologic. To facilitate (b), the pore surfaces of the chromatography medium are functionalized with ligands that bind the impurities with high binding strength, so as to retain them irreversibly once bound. For example, the ligands bind to the impurities with a dissociation constant, K_{d}, in the range of about 10⁻³ M to about 10⁻⁹ M.

In some embodiments, the chromatography medium comprises a plurality of beads or particles having pores, wherein the plurality of beads or particles share a defined pore diameter distribution, and one or more ligands conjugated to the surface of the pores.

In some embodiments, the defined pore diameter distribution is narrow. A narrow pore diameter distribution, as described mathematically elsewhere herein, is one that consistently prevents all the molecules whose hydrodynamic radius is above an assigned cut-off value from accessing the pores, while consistently allowing all the molecules whose hydrodynamic radius is below that assigned cut-off value to access the pores.

In some embodiments, the pore diameter distribution is configured to exclude a target biologic. For example, the pore diameter distribution is selected as a range in which the target biologic would be substantially excluded from entering the pores based solely on size during operation. Thus, the majority of pores (e.g., more than about 70%) have pore diameters smaller than that of the target biologic. In some embodiments, more than about 70%, more than about 75%, more than about 80%, more than about 85%, more than about 90%, or more than about 95% of pores have pore diameters smaller than that of the target biologic. In some embodiments, between about 65% and about 75%, between about 70% and about 80%, between about 75% and about 85%, between about 80% and about 90%, or between about 85% and about 95% of pores have pore diameters smaller than that of the target biologic. In some embodiments, between about 67% and about 73%, between about 72% and about 78%, between about 77% and about 83%, between about 82% and about 88%, between about 87% and about 93%, or between about 92% and about 97% of pores have pore diameters smaller than that of the target biologic. In some embodiments, about 70%, about 75%, about 80%, about 85%, about 90%, or about 95% of pores have pore diameters smaller than that of the target biologic. In some embodiments, more than 70%, more than 75%, more than 80%, more than 85%, more than 90%, or more than 95% of pores have pore diameters smaller than that of the target biologic. In some embodiments, between 65% and 75%, between 70% and 80%, between 75% and 85%, between 80% and 90%, or between 85% and 95% of pores have pore diameters smaller than that of the target biologic. In some embodiments, between 67% and 73%, between 72% and 78%, between 77% and 83%, between 82% and 88%, between 87% and 93%, or between 92% and 97% of pores have pore diameters smaller than that of the target biologic. In some embodiments 70%, 75%, 80%, 85%, 90%, or 95% of pores have pore diameters smaller than that of the target biologic.

Accordingly, the average pore diameter of the pore diameter distribution can be configured and selected based on the size of the intended target biologic. In some embodiments, the average pore diameter is between about 50 Å and about 1000 Å. In some embodiments, the average pore diameter is between about 50 Å and about 150 Å, between about 100 Å and about 200 Å, between about 150 Å and about 250 Å, between about 200 Å and about 300 Å, between about 250 Å and about 350 Å, between about 300 Å and about 400 Å, between about 350 Å and about 450 Å, between about 400 Å and about 500 Å, between about 450 Å and about 550 Å, between about 500 Å and about 600 Å, between about 550 Å and about 650 Å, between about 600 Å and about 700 Å, between about 650 Å and about 750 Å, between about 700 Å and about 800 Å, between about 750 Å and about 850 Å, between about 800 Å and about 900 Å, between about 850 Å and about 950 Å, or between about 900 Å and about 1000 Å. In some embodiments, the average pore diameter is between 50 Å and 150 Å, between 100 Å and 200 Å, between 150 Å and 250 Å, between 200 Å and 300 Å, between 250 Å and 350 Å, between 300 Å and 400 Å, between 350 Å and 450 Å, between 400 Å and 500 Å, between 450 Å and 550 Å, between 500 Å and 600 Å, between 550 Å and 650 Å, between 600 Å and 700 Å, between 650 Å and 750 Å, between 700 Å and 800 Å, between 750 Å and 850 Å, between 800 Å and 900 Å, between 850 Å and 950 Å, between 900 Å and 1000 Å, or between 950 Å and 1050 Å.

In some embodiments, the average pore diameter is more than about 50 Å, about 100 Å, about 150 Å, about 200 Å, about 250 Å, about 300 Å, about 350 Å, about 400 Å, about 450 Å, about 500 Å, about 550 Å, about 600 Å, about 650 Å, about 700 Å, about 750 Å, about 800 Å, about 850 Å, about 900 Å, about 950 Å, or about 1000 Å. In some embodiments, the average pore diameter is more than 50 Å, 100 Å, 150 Å, 200 Å, 250 Å, 300 Å, 350 Å, 400 Å, 450 Å, 500 Å, 550 Å, 600 Å, 650 Å, 700 Å, 750 Å, 800 Å, 850 Å, 900 Å, 950 Å, or 1000 Å. In some embodiments, the average pore diameter may be about 50 Å, about 100 Å, about 150 Å, about 200 Å, about 250 Å, about 300 Å, about 350 Å, about 400 Å, about 450 Å, about 500 Å, about 550 Å, about 600 Å, about 650 Å, about 700 Å, about 750 Å, about 800 Å, about 850 Å, about 900 Å, about 950 Å, or about 1000 Å. In some embodiments, the average pore diameter may be 50 Å, 100 Å, 150 Å, 200 A, 250 Å, 300 Å, 350 Å, 400 Å, 450 Å, 500 Å, 550 Å, 600 Å, 650 Å, 700 Å, 750 Å, 800 Å, 850 Å, 900 Å, 950 Å, or 1000 Å.

In some embodiments, the pore diameter distribution is monodisperse. The dispersity is given by the d₉₀/d₁₀ value which indicates the width of the pore diameter distribution. Here d₉₀ is the value below which 90% of the distribution lies, with d₁₀ corresponding to 10% of the distribution. As the pore diameter distribution for the plurality of beads or particles moves towards monodisperse then the d₉₀ and d₁₀ values become closer together and the d₉₀/d₁₀ value tends towards a value of 1. A pore diameter distribution with a d₉₀/d₁₀ value smaller than about 1.5 is considered a well monodisperse distribution. A pore diameter distribution with a d₉₀/d₁₀ value smaller than about 1.15 is considered a highly monodisperse distribution. For reference, size-exclusion chromatographic resins feature d₉₀/d₁₀ values smaller than 3.0.

The beads and particles can be composed of any materials which facilitate controlling of pore size during production and, ideally, all conjugation of one or more ligands to the surface of the pores, and have a high rigidity to limit the compression and increase the flow velocities with which it can be used. In some embodiments, the plurality of beads or particles are composed of silica, polyacrylamide, polymethacrylate, polystyrene, polyvinylether, or combinations thereof. In select embodiments, the plurality of beads or particles are composed of silica.

The beads and particles may be of various particle sizes selected due to a variety of factors, including, but not limited to, nature of the target biologic, nature of the impurities, method of chromatography being completed (e.g., flow rate, column type and size, etc.), type of ligand, desired pore size, and the like. In some embodiments, the plurality of beads or particles have a particle size ranging between 20 to 150 μm. For example, the particle size may be about 20 μm, about 30 μm, about 40 μm, about 50 μm, about 60 μm, about 70 μm, about 80 μm, about 90 μm, about 100 μm, about 110 μm, about 120 μm, about 130 μm, about 130 μm, or about 50 μm. The particle size may be 20 μm, 30 μm, 40 μm, 50 μm, 60 μm, 70 μm, 80 μm, 90 μm, 100 μm, 110 μm, 120 μm, 130 μm, 130 μm, or 50 μm.

A wide variety of ligands can be attached to beads or particles and used for effective removal of impurities for a sample comprising the target biologic. For example, the one or more ligands may be configured to bind or associate with at least one impurity entering the pore of the bead or particle.

The ligands may be any type of molecule; a small molecule, a biomolecule (e.g., a nucleic acid, an oligonucleotide, a polynucleotide, an amino acid, a peptide, a protein, a monosaccharide, an oligosaccharide, a polysaccharide, a lipid, a steroid, or a conjugate thereof, such as a glycopeptide, a lipopeptide, a glycolipid, a lipoglycopeptide), a chelated or immobilized metal ion, and the like. Exemplary ligands include, but are not limited to, ion exchange ligands, hydrophobic interaction ligands, hydrophilic interaction ligands, polar ligands, thiophilic interaction ligands, affinity ligands, Van der Waals ligands, and combinations of the aforementioned (referred to as mixed mode). Ion exchange ligands include both positively and negatively charged ligands. Exemplary ion exchange ligands include, but are not limited to, sulfate, sulfonate, phosphate, phosphonate, carboxylate; primary, secondary, tertiary amine and quaternary ammonium; heterocyclic amines, such as pyridine, pyrimidine, pyridinium, piperazine, and the like. Hydrophobic ligands are capable of hydrophobic interactions. Exemplary hydrophobic ligands are alkyl, cycloalkyl, haloalkyl, fluoroalkyl, aryl, and the like. Polar ligands include a wide variety of chemical entities comprising polarized chemical bonds, such C—O, C═O, C—N, C═N, C—N, NH, O—H, C—F, C—Cl, C—Br, C—S, S—H, S—O, S═O, C—P, P—O, P═O, P—H. Exemplary polar ligands are carbonyl, carboxyl, hydroxyl, thiol, amide, halide, amine, ester, ether, thioester, and the like. Affinity binding groups may possess any of several binding functionalities that alone or in combination provide a highly specific interaction with target protein. For example, affinity ligands include metal affinity ligands, biological affinity ligands, and immunoaffinity ligands. Biological affinity ligands are those which mediate an interaction with the target biologic based on a biological interaction (e.g., interaction between ligand and protein, or interactions between lectins and glycoconjugates, glycans, glycopeptides, polysaccharides, and other cellular components). Immunoaffinity ligands are those interactions between antigen-antibody or fragments thereof. Metal affinity ligands are those with chelate a metal (e.g., nitriloacetic acid or iminodiacetic acid) or immobilize metal chelates.

The chromatography media may comprise beads or particles having one or more ligands conjugated to a single bead. For example, the chromatography media may comprise beads or particles having one or more ligands conjugated to a single bead or two or more ligands conjugated to the bead as a single group, such as in the case of mixed-mode ligands. Alternatively, the plurality of beads or particles may be comprised of two or more populations of beads or particles which differ only by the type of conjugated ligand. Thus, the medium may comprise two or more populations of beads carrying differing separation or capture functionalities.

The chromatography media may comprise a monolith having one or more ligands conjugated to a single bead. For example, the chromatography media may comprise one or more ligands conjugated to a monolith or two or more ligands conjugated to a monolith as a single group, such as in the case of mixed-mode ligands.

A variety of methods known in the art and those described herein can be used for attaching ligands to a chromatographic medium, including for example direct modification of the bead or particle or monolith with the ligand through suitable coupling chemistry. In general, the criteria of successful attachment include achievement of the desired ligand density and low rate of detachment of the ligand (e.g., low leaching of ligand).

The ligands may be conjugated to the beads using a linker or a spacer. In some embodiments, the linker or spacer facilitate display of the ligand to allow for more efficient capture of the impurities once it enters the pore. In some embodiments, the linker comprises additional functionalities which promote capture of the impurity and increase overall binding of the impurity to the ligand. The choice of linker or spacer will depend on the type of ligand. For example, a peptide-based ligand may utilize a flexible amino acid based linker or spacer, such as those comprised of glycine and serine residues. Alternatively, a peptide-based ligand may utilize a flexible polyethylene glycol (PEG) based linker or spacer. Alternatively, small molecule-based ligands could utilize a linker or spacer which includes functional groups to facilitate conjugation to the bead or particle.

The target biologic can be any suitable biological target. For example, the target biologic may be a polypeptide, a protein, an oligonucleotide, a polynucleotide, a virus or a viral capsid, a portion of the viral capsid, a cell or a cell organelle, or a small molecule. In some embodiments, the target biologic is a protein, such as an antibody, an antibody fragment, an antibody-drug conjugate, a drug-antibody fragment conjugate, a Fc-fusion protein, a hormone, an anticoagulant, a blood coagulation factor, a growth factor, a morphogenic protein, a therapeutic enzyme, an engineered protein scaffold, an interferon, an interleukin, or a cytokine.

As would be understood by one of ordinary skill in the art based on the present disclosure, the target biologic can be any protein, peptide, or polypeptide produced in a cell, including any endogenous, exogenous, or recombinant proteins produced by a cell, and the methods and compositions described herein can facilitate their purification from both process- and product-related impurities.

In other embodiments, the target biologic can be a virus, viral capsid, or viral vector propagated in a cell. In some embodiments, such viruses, viral capsids, or viral vectors are engineered to deliver genetic material into cells for gene therapy, oncolytic applications, or vaccination; therefore, the various embodiments of the present disclosure can be used to purify the target biologic viruses, viral capsids, or viral vectors before they are administered to a cell or a subject. For example, as described further herein, the target biologic can be a retrovirus (RV), such as a lentivirus, an adenovirus (AV), an adeno-associated virus (AAV), a baculovirus, or a herpes simplex virus (HSV). As would be understood by one of ordinary skill in the art based on the present disclosure, the target biologic can be any viral vector produced in a cell, and the methods and compositions described herein can facilitate their purification from both process- and product-related impurities.

In accordance with these embodiments, the chromatography media of the present disclosure can be used in the production of any biologic, including but not limited to, biologic molecules such as antibodies and antibody fragments (e.g., single-chain variable fragments (scFv), single-chain antibodies (scAb), and fragment antigen binding molecules (Fab fragments), diabodies, glycoengineered antibodies, bi-specific antibodies, antibody-drug conjugates, as well as any combinations, derivatives, variants, and fusions thereof. For example, the peptide compositions of the present disclosure can be used to purify any of the currently available therapeutic antibodies, including but not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), infliximab, and bevacizumab.

The one or more product- and/or process-related impurities or contaminants can be any protein, peptide, polypeptide, and/or nucleic acid that is not desirable in a purified composition comprising a target biologic. Product- and process-related impurities also include nutrients that are provided to the cells in a bioreactor (e.g., vitamins, antibiotics, mono/oligo-saccharides, peptide digests, antifoam, surfactants/shear-protecting agents, etc.). For example, product-related impurities can include any fragments or aggregates of the target biologic that are not desired in a purified composition. In other embodiments, the product-related impurities can include an intact target biologic that has undergone a chemical or biochemical modification (e.g., enzyme modification of the amino acid sequence of the target biologic or its profile of post-translational modifications), or an intact target biologic that has become associated with an impurity (e.g., has been rendered inactive).

In some embodiments, the chromatographic media is suspended in solution, packed in a column, or incorporated into a device. In some embodiments, the chromatographic media is a porous monolith or in the form of porous particles. As such, also provided herein are devices comprising the chromatography media described herein. Suitable devices include, for example, columns, cartridges, capsules, and pods. Chromatography columns can be produced from a number of suitable materials, such as glass, metal, ceramic, and plastic. These columns can be packed with the chromatographic media by the end user, or can also be pre-packed with the chromatographic media by a manufacturer and shipped to the end user in a packed state. Thus, chromatography columns and devices pre-packed with the disclosed chromatography media are also provided.

### 3. Methods for Purifying a Target Biologic

The present disclosure also provides improved methods for purifying a target biologic from a sample comprising one or more product- and/or process-related impurities or contaminants, as compared to currently used methods. In some embodiments, the method includes contacting a sample comprising the target biologic to a multifunctional chromatography media as described herein and collecting the target biologic.

In accordance with some embodiments of the chromatography media, the at least one ligand binds or associates with at least one impurity in the sample, allowing the target biologic to be separated from the at least one impurity. The binding may be specific to a particular known characteristic of the impurity or may be a non-specific capture ligand.

In some embodiments, the at least one impurity comprises a fragment of the target biologic, and the target biologic is separated from the fragments of the target biologic. For example, the at least one impurity may comprise a fragment of an antibody, which is separated from the full-length antibody of interest by the disclosed chromatography media due to the size of the fragment allowing it to enter the pores of the media where it is retained or captured due to a binding or association with the at least one ligand on the surface of the pores.

In some embodiments, the at least one impurity is part of an aggregate comprising the target and the at least one impurity is partitioned away from the aggregate due to the high binding strength of the ligand for the at least one impurity as compared to the weaker binding of the impurity to the other components of the aggregate (e.g., the target biologic or other impurities). Thereby the target biologic in the aggregate is purified from the impurity, and in some cases, the aggregate is separated into constituent components.

The binding affinity of the chromatography media for the impurities, as compared to the target biologic, can be altered by changes in the composition, concentration, and pH of the sample and/or the loading conditions and residence time of the contacting and washing steps. Any of these variables can be changed to variables which are suitable according to the methods of the present disclosure and result in the desired binding affinity for the particular target biologic and impurity.

The methods of the present disclosure can further comprise washing the chromatography medium to remove one or more unbound target biologics into a wash fraction; and then collecting the wash fraction containing the one or more unbound target biologics.

In some embodiments, the method can be performed under any binding conditions suitable for use with the chromatography medium, including both static binding conditions and dynamic binding conditions. In some embodiments the unbound target biologics are collected into an unbound fraction and/or wash fraction when the methods are performed under static binding conditions. In some embodiments the unbound target biologics are collected into a flow through or a column wash fraction when the methods are performed under dynamic binding conditions.

As would be appreciated by one of ordinary skill in the art based on the present disclosure, the methods described herein can be used before or after any purification method typically used to purify and/or isolate a given target biologic. For example, the methods disclosed herein can be used before or after: ion exchange chromatography (e.g., cation exchange chromatography, anion exchange chromatography), affinity chromatography (e.g., Protein A affinity chromatography), size exclusion chromatography, mixed mode chromatography, hydrophobic interaction chromatography, hydrophobic charge induction chromatography, hydroxyapatite chromatography, another chromatography step using a chromatography media as disclosed herein, and/or a filtration treatment.

In some embodiments, the methods disclosed herein are used after a cell culture fluid has been clarified, but prior to performing a chromatography step. In select embodiments, the methods disclosed herein are used after a cell culture fluid has been clarified, but prior to performing primary capture chromatography (e.g., Protein A affinity chromatography). In some embodiments, the methods disclosed herein are used after a cell culture fluid has been clarified and at least one other chromatography step. In select embodiments, the methods disclosed herein are used after a cell culture fluid clarification and a primary capture chromatography (e.g., Protein A affinity chromatography). For example, in some embodiments, the sample comprising the target biologic is prepared by carrying out an affinity chromatography step and collecting an eluate comprising the target biologic to act as the sample for the methods disclosed herein.

In some embodiments, the methods disclosed herein are used as a single step to purify the target biologic (e.g., following cell culture fluid clarification).

In some embodiments, the methods of the present disclosure are particularly suitable for use in the manufacturing of therapeutic antibodies, which can greatly benefit from the adoption of the chromatography media of the present disclosure owing to their potential of transforming the downstream process from a pipeline of “batch” chromatographic steps operated in “bind-and-elute” mode to a pipeline of continuous and connected chromatographic train operated in flow-through” mode. However, the methods described herein are also applicable to the purification of other target biologics, such as gene therapy products. These include, for example, viruses for in vivo (e.g., adenovirus and adeno-associated virus) and in vitro (e.g., lentivirus and baculovirus) gene therapy.

The compositions and methods of the present disclosure can support the production of non-therapeutic proteins, for example for the biotech/ag-bio industry. Improvement of livestock and crops via genetic engineering (e.g., CRISPR) requires the availability of purified gene-editing enzymes (e.g., Cas9 nucleases). Very often, these proteins feature remarkable biochemical lability, which makes large-scale purification challenging and limits product throughput and quality, thereby increasing drastically the price of these products. Accelerating and simplifying the purification process of these products is an essential contribution towards enabling their widespread use in the future.

The chromatography media can be utilized in any conventional configuration, including packed columns and fluidized or expanded-bed columns, and by any conventional method. The use of a packed flow-through column is particularly convenient, both for preparative-scale and analytical-scale.

Embodiments of the present disclosure also provide for methods for selecting the chromatography medium which would facilitate separation of a target biologic from one or more impurities in a single step. Selection of the chromatography medium is based on two or more characteristics of the one or more impurities in a sample and the target biologic. As described above, the impurities can be product- or process-related impurities. In some embodiments, the methods include determining a molecular weight or size of the impurities and at least one additional characteristic for the impurity (e.g., a characteristics which is different between the target biologic and the impurity). Exemplary characteristics include, but are not limited to, charge, phosphorylation state, hydrophobicity, polarity, affinity, glycosylation state, and the like.

Experimental determination of the molecular weight or size of an impurity can be accomplished with routine methods including mass spectrometry (e.g. matrix-assisted laser desorption ionization—time of flight mass spectrometry, MALDI-TOF MS), size exclusion chromatography, gel electrophoresis, analytical ultracentrifugation, light scattering. Identifying one or more additional characteristic is completed using any variety of experimental methods depending on the particular characteristic being examined: charge is determined via isoelectro focusing gel electrophoresis and measurement of zeta potential; hydrophobicity is evaluated by measuring the retention time in a reverse phase chromatographic column (e.g., C8 or C18) or relative retention time in a hydrophobic interaction chromatography (HIC) analytical column vs. a standard of known hydrophobicity; protein folding is evaluated via circular dichroism and functional assays (e.g., ELISA or Western blotting).

The medium is selected such that the target biologic is excluded based on molecular weight or size. For example, the multifunctional chromatography medium is chosen such that the plurality of beads or particles have pores with a diameter smaller than the molecular weight or size of the target biologic. In another example, the multifunctional chromatography medium is chosen such that the monolith has pores with a diameter smaller than the molecular weight or size of the target biologic. Additionally, the multifunctional chromatography medium is chosen such that the plurality of beads or particles have pores with a diameter which are larger than the molecular weight or size of the impurities, such that the impurity is able to enter the pores.

The medium is further selected such that it has one or more ligands functionalized to surfaces of the pores based on the additional characteristic, such that the impurities upon entering the pores will bind or associate with the ligands and be trapped, thus separating the impurity from the target biologic.

### 4. EXAMPLES

The accompanying Examples are offered as illustrative as a partial scope and particular embodiments of the disclosure and are not meant to be limiting of the scope of the disclosure.

The future biologics manufacturing will most likely rely on next-generation chromatography media that are also conducive for continuous operation, with an improved capability of removing impurities, especially high risk species (e.g., high risk host cell proteins) that have been identified as persistent in current bioprocesses. For example, the platform downstream process of antibody purification supplies a multitude of products in large quantity and high purity to clinics worldwide every year. While being revised over the years, the process maintains its core framework, which starts with an affinity-based capture step, typically employing Protein A resins, and proceeds through a series of product polishing steps. These are tasked with removing residual product- and process-related impurities after the capture step, and are currently performed with ion-exchange (IEX) and mixed-mode (MM) resins. Conducting product polishing in flow-through mode has gained considerable interest in the biopharmaceutical industry, being conducive to continuous operation and process intensification. Flow-through chromatography requires judicious selection of process conditions to ensure efficient capture of the whole spectrum of impurities, while minimizing capture of the product and accounting for variability in the feed stream. Current commercial chromatography media rely solely on the protein-ligand interaction, and therefore on the composition and pH of the binding environment, as a means to achieve the necessary orthogonality of target vs. impurity binding.

As shown herein, the selection activity of various ligands with the size-exclusion activity of silica resins featuring highly mono-dispersed pore diameter achieves efficient and robust polishing in flow-through mode. Optimal pore size can indeed exclude the target biologic, e.g., antibody, while allowing impurities, such as fragments of an antibody, to access the resins, whereupon they are captured by the MM ligands. The exclusion of the target biologic can (i) broaden substantially the space of operation—namely, composition, concentration, and pH of the mobile phase—that grants high yield and purity and (ii) reduce the complexity of process optimization for polishing with different properties (e.g., charge and amphiphilicity).

### Example 1

### Design of Mixed-Mode Size-Exclusion Silica (SEMM-Silica) Resins

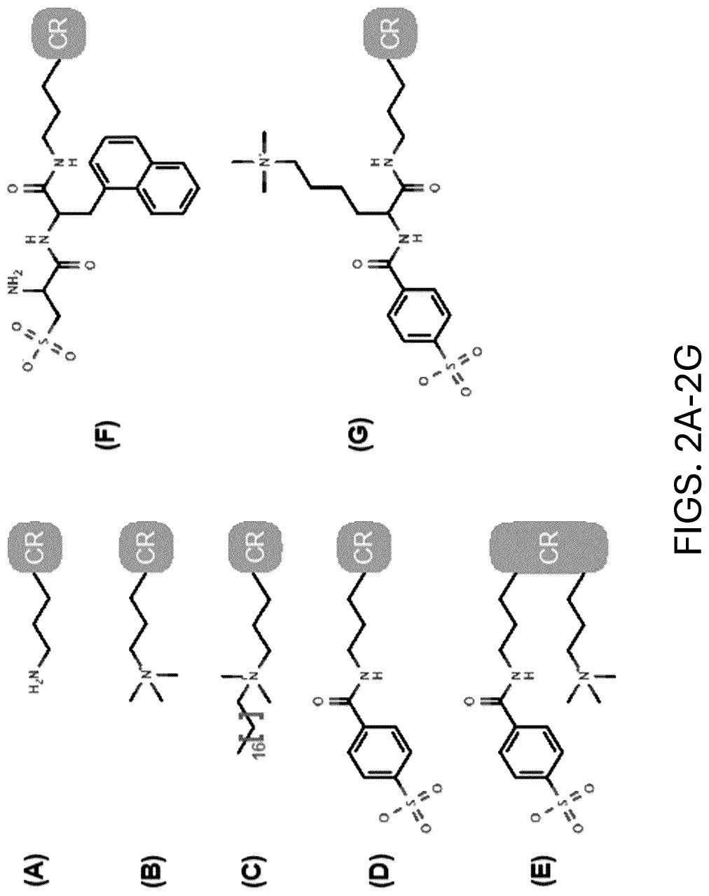

The mixed-mode (MM) ligands in this study were designed with the intent of interrogating the various interactions that contribute to the MM-type binding: primary and quaternary amines to evaluate the effect of strength of cation-exchange binding (FIGS. 2A and 2B); an alkyl chain displayed on a quaternary amine to evaluate the combined effect of cation-exchange and hydrophobic interactions (FIG. 2C); and (4-sulfonatophenyl)amide, naphthyl-alaninyl cysteamide, and trimethyl-lysyl sulfobenzoamide groups to compare the combined effect of weak vs. strong anion- and cation-exchange, soft vs. strong aromatic, and hydrogen bond interactions (FIGS. 2D, 2F, and 2G).

The ligands were conjugated onto Chromatorex silica resins featuring a fixed particle diameter (65 μm) and varying pore diameter (100, 200, or 300 Å). The value of particle diameter was not varied. Since silica beads are practically incompressible, the permeability of the packed bead is constant and the values of pressure drops recorded in this study at different linear velocities and column lengths were within the acceptable range. Pore diameter values were selected based on the characteristic molecular dimensions of IgG (145 Å×85 Å×40 Å, [24])—and its F(ab)₂ (75 Å×35 Å×25 Å,), Fc (70 Å×65 Å×25 Å,), and Fab (70 Å×55 Å×25 Å,) fragments. Accordingly, it was assumed that values of pore diameter below 100 Å would reduce product purity by hindering the capture of larger IgG fragments (e.g., Fab₂ and clipped forms), whereas values above 300 Å would reduce product yield by enabling access and capture of IgG in the pores.

Based on the design of the SEMM-silica resin and their application task, five critical process parameters, namely ligand identity, pore size, linear velocity (residence time), composition and pH of the mobile phase, and ionic strength of the feedstock, were evaluated in sequence.

### Example 2

### Selection of Pore Size Via Static Binding Isotherms

To select the value of pore size static binding studies were conducted using monomeric IgG and its fragments (Fc and Fab). The values of equilibrium adsorption, in fact, reveal whether a resin has an inherent propensity to bind full IgG, thus causing a yield loss, or an insufficient ability to capture its fragments, thus failing to afford the desired purity. Accordingly, binding isotherm curves were constructed by incubating APTES-Chromatorex, TMTAC-Chromatorex, and DTS-Chromatorex resin—each with pore size of either 100, 200, or 300 Å—in solutions of human IgG with titer ranging up to 40 mg per mL. The three resins were adopted for this study to ensure the selection of a pore size value that minimizes full IgG binding irrespective of the binding strength of the ligands (DTS>TMTMAC>APTES). Two values of pH, 7 and 8, were explored to probe the effect of net IgG charge on its interaction with the ligands. The adsorption datapoints recorded at low concentration provided reliable values of the dissociation constant (KD), while those recorded at high concentration encompassed the IgG titer in the eluates of Protein A resins (5-30 grams per liter) and were intended to provide the values of maximum binding capacity (Qmax).

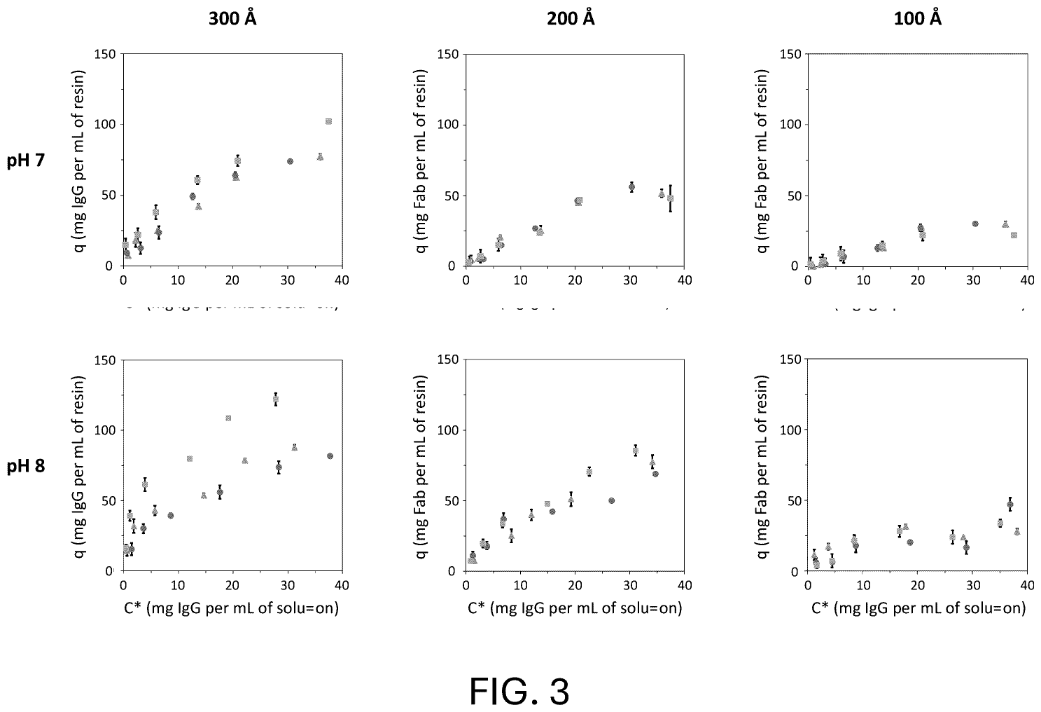

The resultant adsorption isotherms reported in FIG. 3 indicated that (i) lowering the pore size decreases the binding capacity as well as the difference in IgG adsorbed by the different ligands; and (ii) the IgG capture grows up to 1.5-fold as the pH increases from 7 to 8. The values of Qmax and KD obtained by fitting the adsorption data against a Langmuir isotherm ad-sorption model are reported in Table 1. These trends were anticipated: as the pore diameter approaches the hydrodynamic radius of IgG, it obstructs the protein's access to the silica beads; as the pH increases, the IgG surface acquires a stronger anionic character and interacts more strongly with the cationic MM ligands.

Notably, the adsorption data obtained using TMTAC- and DTS-Chromatorex resins with 300 Å pore diameter presented a trend that combines the Langmuir and Freundlich isotherms: the amount of adsorbed IgG increases steeply with the equilibrium concentration at low IgG titer, indicating strong binding by the MM ligands; at higher IgG titer in solution, the adsorbed IgG increases linearly, suggesting the formation of a protein multilayer.

The SEMM-silica resins with pore diameter of 200 Å and 100 Å presented a Langmuir-only trend at both values of pH. The reduction in pore diameter may have prevented the formation of an IgG multilayer on the pore surface, thereby removing the Freundlich-like trend from the adsorption data recorded at higher IgG titer. TMTAC- and DTS-Chromatorex resins with 200 Å pore diameter featured Qmax ranging between 80 and 120 grams per liter, in line with commercial strong ion-exchange resin, which reach values of up to 100 grams of IgG per liter of resin. Conversely, the resins with 100 Å pore diameter exhibited a significantly lower Qmax between 10 and 30 grams per liter, suggesting their use for fragment capture in flow-through mode.

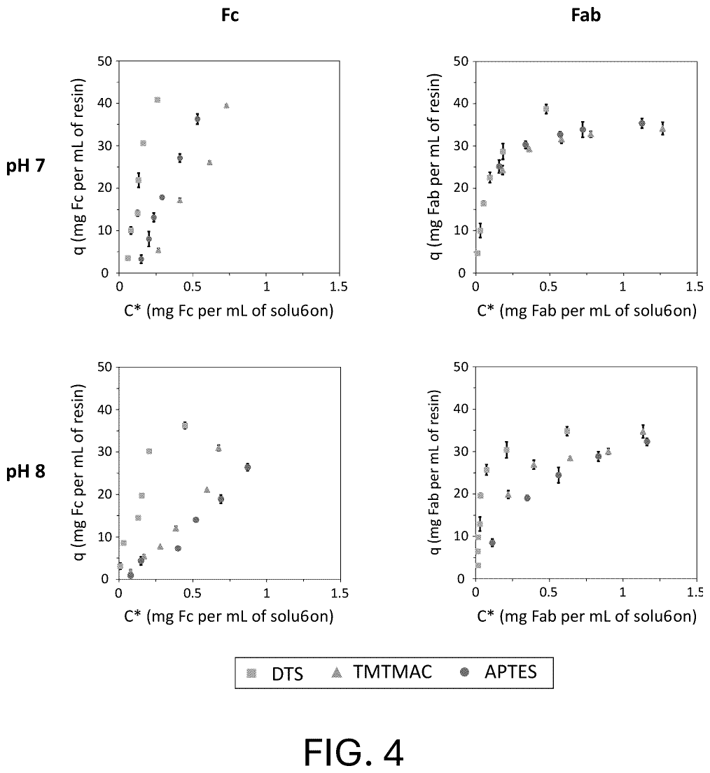

Subsequently, the static binding of IgG fragments Fc and Fab was evaluated on SEMM-silica resins with 100 Å pore diameter (FIG. 4); the values of Qₘₐₓ and K_{D} obtained by fitting the adsorption data against a Langmuir isotherm adsorption model are reported in Table 2. The values of concentrations of F(ab)₂, Fc, and Fab utilized to build the isotherms ranged between 0.25 and 5 grams per liter, which encompass the range observed in Protein A eluates (e.g., from as low as 2% to as high as 20% w/w fragments). As observed with monomeric IgG, the capture of fragments increased with the binding strength of the ligands. Whilst values of Qₘₐₓ could not be derived since the isotherms were constructed using diluted feedstocks, the adsorption data demonstrated that the fragments are efficiently captured by DTS-silica resin. Collectively, the results of the static binding studies indicate that silica resin with 100 Å pore diameter are conducive to high values of IgG yield and monomeric purity when operated in flow-through mode.

TABLE 1
Values of full IgG equilibrium binding capacity (Qₘₐₓ)
and dissociation constant (K_{D}) calculated by fitting the
adsorption data in FIG. 3 against Langmuir isotherm adsorption
model. The adsorption data were obtained by incubating
solutions of human IgG with titer ranging from 1 to 40
g/L in either 50 mM Phosphate buffer at pH 7 or 50 mM
Tris HCl buffer at pH 8 with APTES-Chromatorex, TMTAC-
Chromatorex, and DTS-Chromatorex resin with pore diameter of
300 Å, 200 Å, or 100 Å.

| | | pH 7 | | pH 8 | |
|---|---|---|---|---|---|
| | Pore Size | Qₘₐₓ | K_{D} | Qₘₐₓ | K_{D} |
| Ligand | (Å) | (mg/mL) | (M) | (mg/mL) | (M) |
| APTES | 300 | † | | † | |
| | 200 | 56 | 9.53 10⁻⁵ | | |
| | 100 | 45 | 2.27 10⁻⁴ | | |
| TMTMAC | 300 | 73 | 2.03 10⁻⁴ | | |
| | 200 | 52 | 2.17 10⁻⁴ | | |
| | 100 | 32 | 2.65 10⁻⁴ | | |
| DTS | 300 | † | | | |
| | 200 | 68 | 1.83 10⁻⁴ | | |
| | 100 | 31 | 2.89 10⁻⁴ | 33 | 2.47 10⁻⁴ |

†: value could not be calculated because the adsorption data did not display a Langmuir-type trend.

TABLE 2
Values of equilibrium binding capacity (Qₘₐₓ) and dissociation
constant (K_{D}) calculated by fitting the adsorption data in FIG.
4 against Langmuir isotherm adsorption model. The adsorption data
were obtained by incubating solutions of human Fc and Fab with
titer ranging from 1 to 5 g/L in either 50 mM Phosphate buffer
at pH 7 or 50 mM Tris HCl buffer at pH 8 with APTES-Chromatorex,
TMTAC-Chromatorex and DTS-Chromatorex resin (pore size: 100 Å).

| | | pH 7 | | pH 8 | |
|---|---|---|---|---|---|
| | | Qₘₐₓ | K_{D} | Qₘₐₓ | K_{D} |
| Ligand | Target | (mg/mL) | (M) | (mg/mL) | (M) |
| APTES | Fc | † | † | † | † |
| | Fab | 18 | 1.22 10⁻⁵ | 20 | 1.86 10⁻⁶ |
| TMTMAC | Fc | † | † | † | † |
| | Fab | 17 | 3.23 10⁻⁶ | 18 | 1.80 10⁻⁶ |
| DTS | Fc | † | † | † | † |
| | Fab | 26 | 7.99 10⁻⁶ | 21 | 2.02 10⁻⁶ |

†: value could not be calculated because the adsorption data did not display a Langmuir-type trend.

### Example 3

### Screening of Resin Pore Size Using Breakthrough Curve Simulation

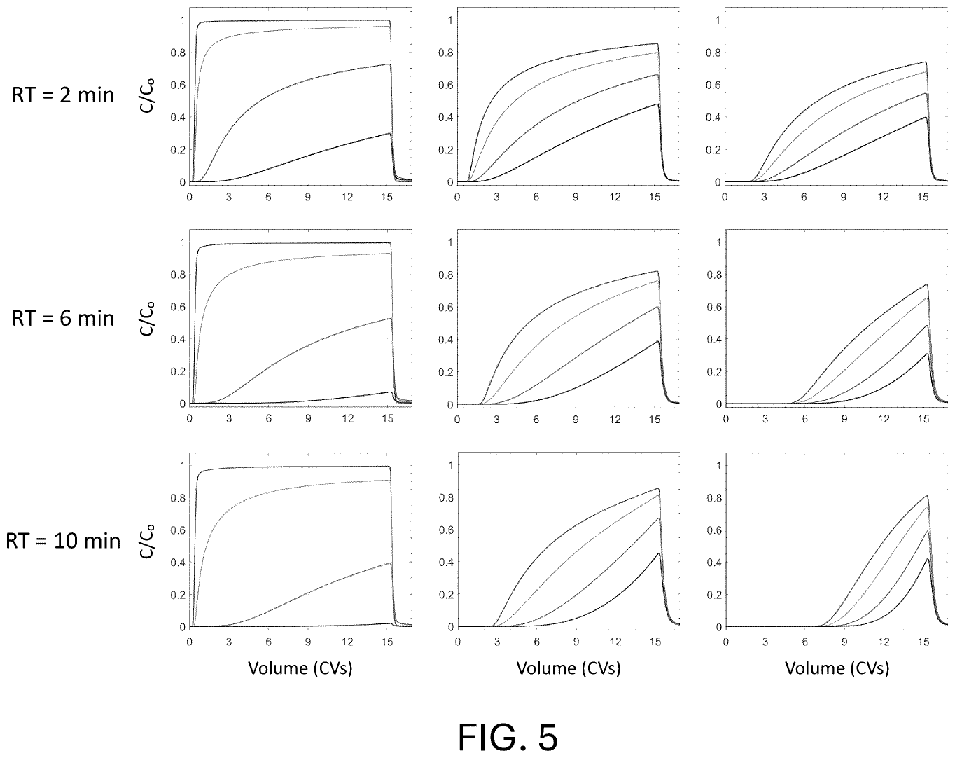

Breakthrough curve simulations were performed using a model feedstock containing monomeric IgG, Fab₂, Fab/Fc, and LMW species with mass fractions of 0.9, 0.03, 0.05, and 0.02, respectively, and an overall protein concentration of 6 mg/mL. The simulations were performed at different values of residence time (RT: 2, 6, and 10 min) and pore diameter of the resin (i.e., 100, 200, and 300 Å) assuming a load volume of 15 CVs and a column wash volume of 2 CVs. The resultant breakthrough curves, collated in FIG. 5, indicate that the concentration of monomeric IgG in the effluent from the adsorbent with 100 Å pore diameter rapidly reached the corresponding value in the feed, indicating that the mAb does not appreciably diffuse into the pores, for all the values of RT. Restricting mAb adsorption to the outer surface of the resin provides full binding capacity to the fragments, which can freely diffuse through the pore space and saturate the resin.

Conversely, resins with larger pore size did not see the mAb concentration reach its plateau value, indicating significant losses in product yield. As for the fragments, significant discrepancies were observed between the shape of the breakthrough curves obtained with pore size of 100 Å versus those obtained with 200 and 300 Å. Collectively, these results corroborate the selection of a pore diameter of 100 Å as the value that provides superior partitioning of fragments vs. intact antibody owing to the difference in the pore diffusion rates between the mAb and the fragments (note: the ratios of the diffusion coefficients of fragments vs. intact antibody, calculated using the values reported in Tables 7-9 are D_{(Fab)2}/D_{IgG}˜47.8, D_{Fc}/D_{IgG} and D_{Fab}/D_{IgG}˜804.6, and D_{LMW}/D_{IgG}˜3871.6). It is worth noting that, while providing better removal of Fab₂, the resins with pore diameters of 200 and 300 Å underperformed significantly in terms of removing all other fragment species. This was especially evident at the RT of 10 min, where Fab/Fc and LMW reached a lower plateau concentration at 15 CVs compared to the resins with larger pore size.

### Example 4

### Ligand Selection Via Small-Loading Dynamic Binding Studies

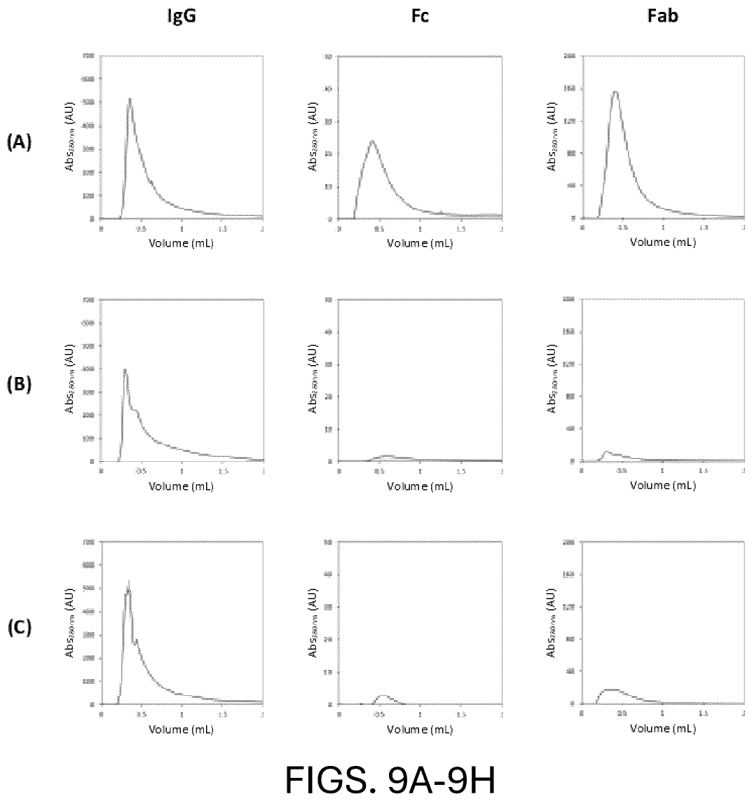

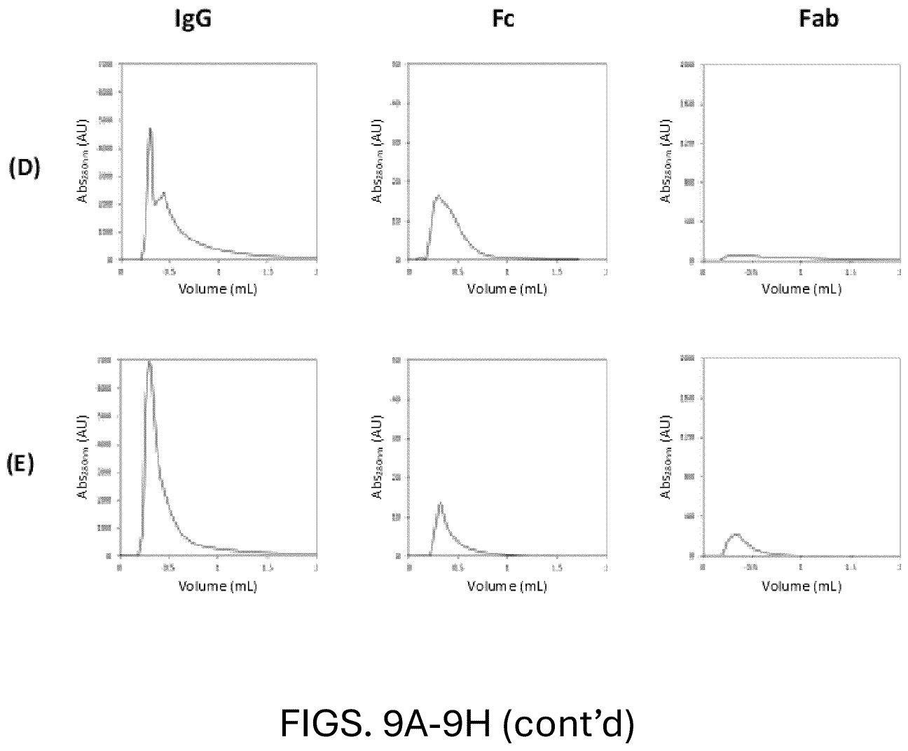

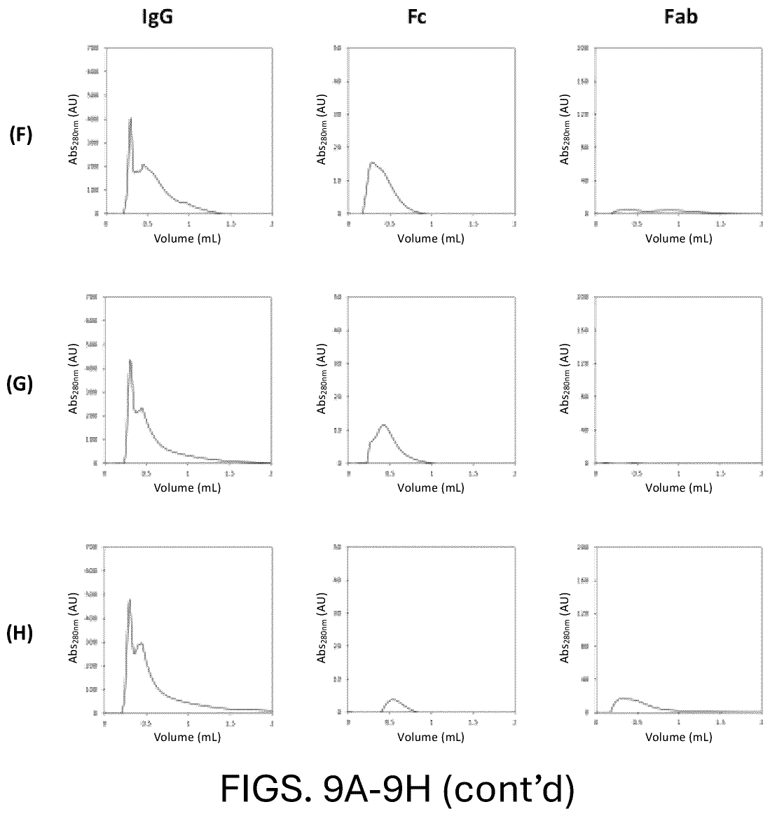

The SEMM-silica resin listed in FIG. 2 were evaluated in dynamic mode by loading a small volume (1 CV) of monomer IgG or fragments. The model feedstocks comprised single-protein solutions of human IgG at 15 g/L and Fc or Fab fragment at 2.5 g/L, which simulate the composition of challenging Protein A eluates (˜10% fragments,). Small-loading dynamic binding studies represent a rigorous approach to evaluate the yield of monomeric IgG, since only the resins that feature little-to-no IgG binding will afford the desired high product yield. Conversely, the loading of fragment solutions at 2.5 g/L, which corresponds to 12.5% w/w of the total protein titer, is ideal to challenge the fragment removal activity of the SEMM-silica resin. Small injection volumes of the feedstocks also allowed for “chasing” the load with a washing solution (i.e., 50 mM phosphate buffer at pH 7) to (i) maximize product yield by displacing the monomeric IgG from the interstitial porosity between the silica beads and (ii) ensure that bound IgG fragments are stably retained by the MM ligands. The resulting chromatograms, reported in FIG. 9, were analyzed to derive the %-values of IgG, Fc, and Fab capture. From these values, a purification score was calculated (P-score, ranges from 0 to 1) using Equation 3 and utilized to rank the performance of the SEMM-silica resin (Table 3). The adsorbents DTS-Chromatorex, TMTMAC-Chromatorex, and APTES-Chromatorex resins featured values of P-score significantly higher than those obtained with the other resins, and were therefore carried forward to subsequent analyses.

TABLE 3
Values of protein capture obtained by loading a solution
of human either IgG (15 g/L) or Fc fragment (2.5 g/L)
or Fab fragment (2.5 g/L) prepared in 50 mM Tris HCl
buffer at pH 8.0 onto DTS-Chromatorex, TMTMAC-Chromatorex, SBA-
Chromatorex, SBA/TMTMAC-Chromatorex, SBA-Lys(Me)₃-
Chromatorex, and CA-1-Nal-Chromatorex, and APTES-Chromatorex
resins (pore diameter: 100 Å) at 173 cm/h.

| | IgG | Fc | Fab | |
|---|---|---|---|---|
| SEMM-silica resin | capture | capture | capture | P-score* |
| TMTMAC-Chromatorex | 5% | 92% | 89% | 0.78 |
| APTES-Chromatorex | 12% | 92% | 84% | 0.68 |
| DTS-Chromatorex | 20% | 89% | 89% | 0.63 |
| CA-1-Nal-Chromatorex | 23% | 66% | 99% | 0.50 |
| SBA-Lys(Me)₃-Chromatorex | 6% | 54% | 92% | 0.46 |
| SBA/TMTMAC- | 8% | 59% | 68% | 0.36 |
| Chromatorex | | | | |
| SBA-Chromatorex | 25% | 53% | 91% | 0.36 |

### Example 5

### Selection of Linear Velocity of the Mobile Phase

As with commercial MM adsorbents, the polishing activity of SEMM-silica depends on the physicochemical properties of the load and the mobile phase “chasing” it. At the same time, the selected pore size of the silica resin puts significant emphasis on the role of the linear velocity: the reduction in transport kinetics of IgG fragments throughout the silica beads imposed by the small pore diameter requires prolonging the residence time of the feedstock into the packed bead to ensure appropriate capture of all fragments. Accordingly, five values of residence times (i.e., 3, 4, 5, 6, and 7 min) and four values of pH (6, 7, 8, and 9) were evaluated using a model Protein A eluates comprising monomeric IgG at 5 g/L, IgG aggregates at 0.35 g/L, Fab₂ at 0.25 g/L, Fc and Fab fragments each at 0.28 g/L, and low molecular weight fragments at 0.02 g/L.

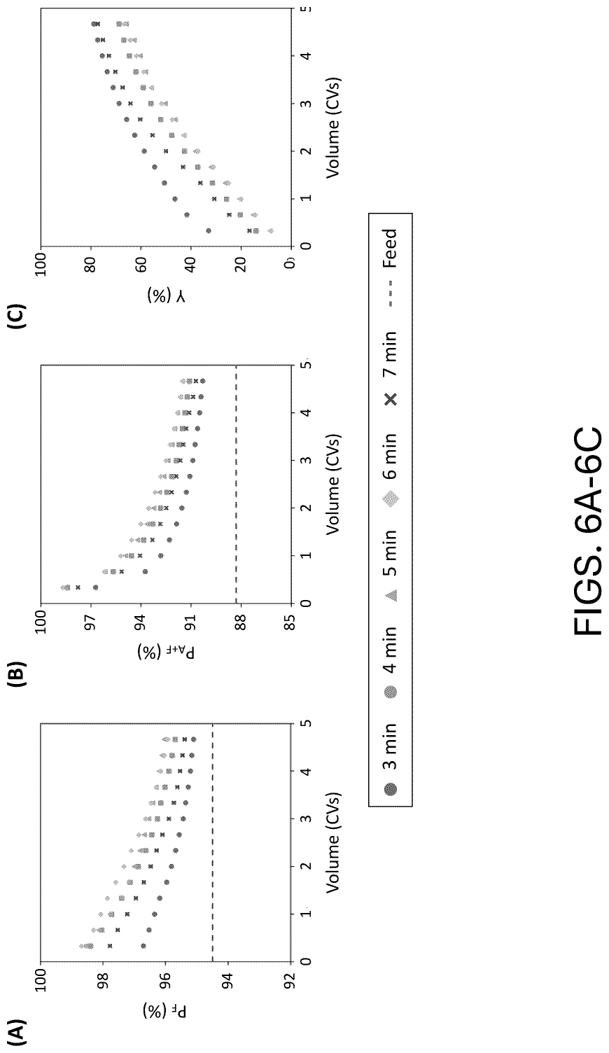

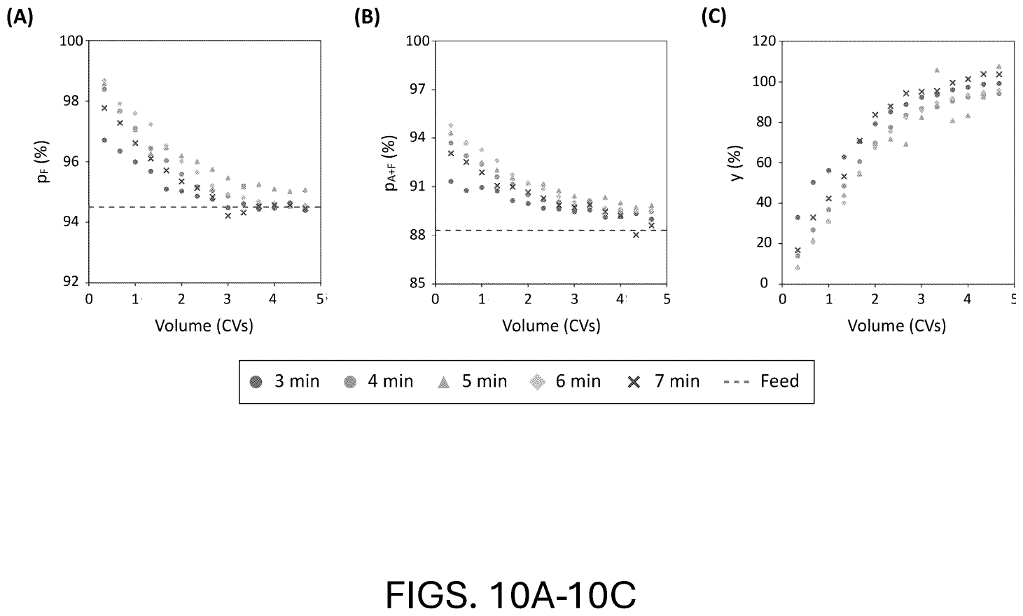

The results of the residence time study on the cumulative values of antibody purity and yield obtained with APTES-Chromatorex resin are presented in FIG. 6 and summarized in Table 4; the corresponding values of fractional monomeric purity are reported in FIG. 10. Residence time of 3 min (corresponding to a linear velocity of 200 cm/h) afforded the highest yield and the lowest monomeric antibody purity. The fast flow rate, in fact, reduced the access of full antibodies to the mouth of the pore, whose diameter is larger than the average value, but it also limited the capture of antibody fragments and aggregates (FIGS. 6A and 6B), which flow through the column resulting in a low purity of the effluent. Increasing the residence time to 7 min (85.7 cm/h) also lowered the monomeric antibody purity, for it promoted the adsorption of full antibodies without increasing the binding of fragments; as observed in the static binding studies, the DTS-Chromatorex resin with pore size of 100 Å features a binding capacity of ˜25 grams of IgG per liter of resin when loaded with a feedstock containing IgG at 5 g/L. The loss of antibody yield can be observed in FIG. 6C, which portrays the temporal profiles of cumulative and fractional yield, respectively. Finally, the intermediate values of residence time afforded similar values of product purity and yield, with 6 min (100 cm/h) providing the best outcome—namely, 92% and 63% upon loading 5 CVs.

A notable result is also represented by the significant reduction of aggregates (FIG. 6B): a portion of these aggregates, especially the larger species that cannot access the pores, is captured on the surface of the resin. The amount of captured aggregates, however, clearly exceeds the capacity of the mere surface of the silica resin; furthermore, if the silica resin particles were to be fully coated by large, pore-blocking species, the effective capture of antibody fragments could not be explained. This is likely due to a significant portion of the aggregates being unstable resulting in disassembly upon adsorbing unto the pore's mouth and release of smaller constituents, including antibody fragments, which access the pores and are captured therein.

TABLE 4
Values of cumulative yield (Yₙ) and purity of monomeric IgG calculated with respect to the capture of IgG
fragments only (P_{F, n}) obtained in the n^{th} fraction (i.e., value of load in column volumes, CVs) at different
values of residence times (RT). The values are extracted from the temporal profiles in FIG. 6.

| | RT | | | | | | | | | |
|---|---|---|---|---|---|---|---|---|---|---|
| | 3 min | | 4 min | | 5 min | | 6 min | | 7 min | |
| Load (n) | Yₙ | P_{F, n} | Yₙ | P_{F, n} | Yₙ | P_{F, n} | Yₙ | P_{F, n} | Yₙ | P_{F, n} |
| 0.7 CVs | 41.6% | 96.5% | 20.4% | 98.0% | 15.2% | 98.1% | 14.4% | 98.3% | 24.8% | 97.5% |
| 1.7 CVs | 54.5% | 96.0% | 37.4% | 97.1% | 32.0% | 97.2% | 31.1% | 97.6% | 43.2% | 96.7% |
| 2.7 CVs | 65.7% | 95.6% | 52.2% | 96.4% | 46.3% | 96.7% | 47.6% | 96.9% | 60.3% | 96.1% |
| 3.7 CVs | 73.4% | 95.3% | 62.0% | 96.0% | 58.1% | 96.3% | 58.9% | 96.3% | 70.2% | 95.6% |
| 4.7 CVs | 78.8% | 95.1% | 68.7% | 95.7% | 65.9% | 96.0% | 66.6% | 95.9% | 77.2% | 95.4% |

### Example 6

### Selection of pH and Conductivity of the Mobile Phase

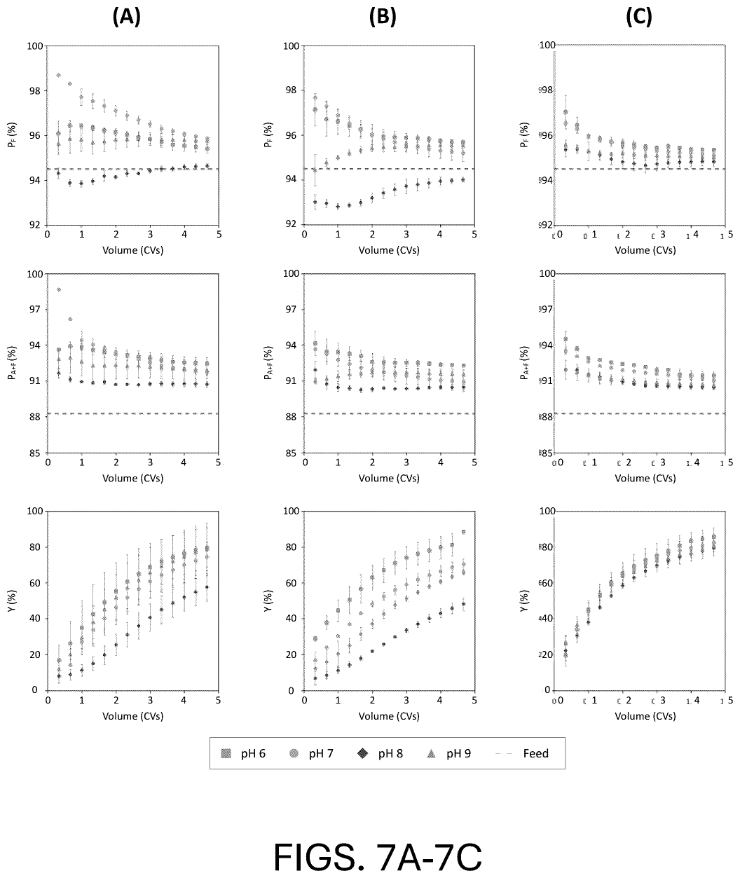

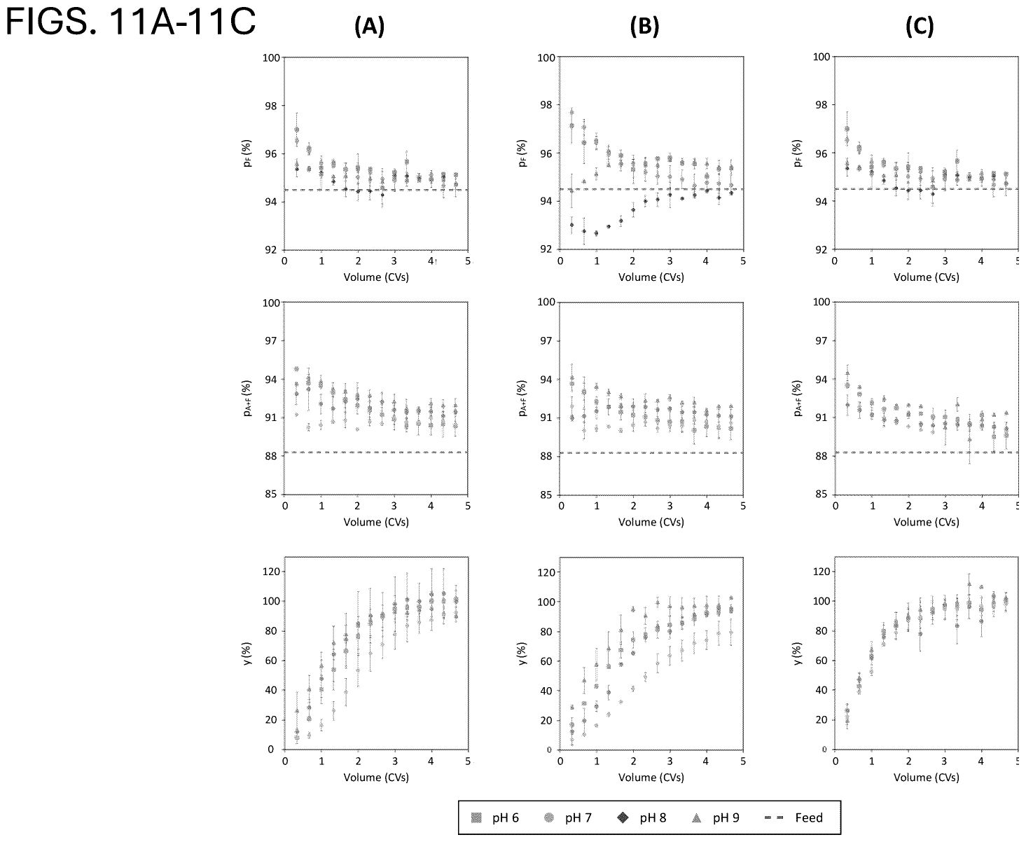

It has been reported recently that pH, followed by conductivity, are the most significant factors determining antibody recovery and monomeric purity. Accordingly, the role of pH and conductivity of both feedstock and column washing (“chasing”) buffers was investigated while keeping the value of residence time constant at 6 min. This choice was made under the assumption that (i) most of the fragment capture occurs inside the resin's pores—i.e., the amount of fragments captured on the surface of the silica particles is negligible; (ii) the amount of fragments that, migrating from the feed to the surface of particles, access their pores depends solely on the linear velocity; and (iii) the fraction of fragments that, having entered the pores, are retained therein depends solely on the fragment:ligand interaction, and thus ligand chemistry and composition of the mobile phase. Therefore, while having used APTES-Chromatorex resin to finalize the value of residence time, the effect of pH and conductivity was investigated on three ligands selected in Example 3, namely APTES, TMTMAC, and DTS. The temporal profiles of cumulative purity and yield of the antibody monomer are presented in FIG. 7, while the corresponding fractional values are reported in FIG. 11. Selected values of purity and yield obtained at different load volume are presented for APTES-Chromatorex resin in Table 5, and for TMTMAC-Chromatorex and DTS-Chromatorex resins in Tables 7 and 8.

Collectively, the results in FIG. 7 indicate that operating antibody polishing under acidic-to-neutral conditions consistently afforded higher values of yield and purity. This may be somewhat unexpected, since the human antibody utilized in this study has an average isoelectric point (pI) of 8 and all three ligands utilized herein feature cationic groups. These results can be explained by examining the behavior of the single SEMM-silica resin. First, APTES-Chromatorex is designed as a standard weak anion-exchange resin, whose primary amine groups (pKₐ˜10.5) are positively charged at all values of pH examined herein. At pH 9, the monomeric antibodies and—even more so their aggregates—present a net negative charge, whereas at pH 6 and 7 both monomeric and aggregated antibodies present a net positive charge. Accordingly, the SEC analysis of the effluents at pH 9 showed an effective capture of the aggregates and a minor retention of monomeric antibodies, which, while unable to access the pores, can be adsorbed on the surface of the silica beads; at pH 6 and 7, the yield of monomeric antibodies increases, but so does the escape of the aggregates, likely due to the electrostatic repulsion. Notably, the SEC results also show that specific groups of fragments flow through at pH 9 and are effectively retained at pH 6 and 7, suggesting that these species bear a net negative charge, opposite to that of their cognate antibody. This in turn provides a rationale for the loss of product purity and yield at pH 8: the anionic antibody fragments that are captured by the amine ligands on the Chromatorex resin can in turn act as binders to the monomeric antibodies; the equivalence of pH and pI drives further antibody binding, resulting in a substantial yield loss.

TMTMAC- and DTS-Chromatorex resins are functionalized with strong anion-exchange quaternary amines. The electrostatic repulsion between the cationic ligands and the monomeric antibodies, could explain the decreased fragment removal—especially at pH 6 and 7—achieved by DTS-Chromatorex and TMTMAC-Chromatorex (FIGS. 7B and 7C); the monomeric antibody yield, however, shows a downward trend as the pH of the load increases, as the progressive appearance of anionic surface regions promotes antibody capture on the surface of the beads. All ligands also promote product purity, in terms of both fragment and aggregates capture: in particular, APTES-Chromatorex resin, owing to its combination of electrostatic and size exclusion ability affords a fragment and aggregate reduction up to 2-fold when loaded with up to 3 CVs of model protein A eluate. For comparison, the adsorbent Capto Adhere and Capto MMC operated in bind-and-elute mode respectively afforded comparable values of 2.3-5.1-fold, demonstrating the ability of SEMM-silica resin to compete with commercial adsorbents for antibody polishing. Notably, the values monomeric antibody purity in all effluents plateau at values higher than the corresponding value in the model Protein A eluate, indicating that the SEMM-silica resins are far from saturated, even when loaded with conspicuous volumes of feedstock.

While improving significantly the antibody purity, the optimization of pH alone does not afford an effluent of sufficient quality to withstand the comparison with industrial processes. Furthermore, as observed in Table 5, product purity decreases rather steeply with the load volume: up to 1 CVs, the monomeric antibody purity is above 98%, but the yield is below 40%; as the load is increased to 5 CVs, where the yield approaches 80%, the purity has decreased to 96%.

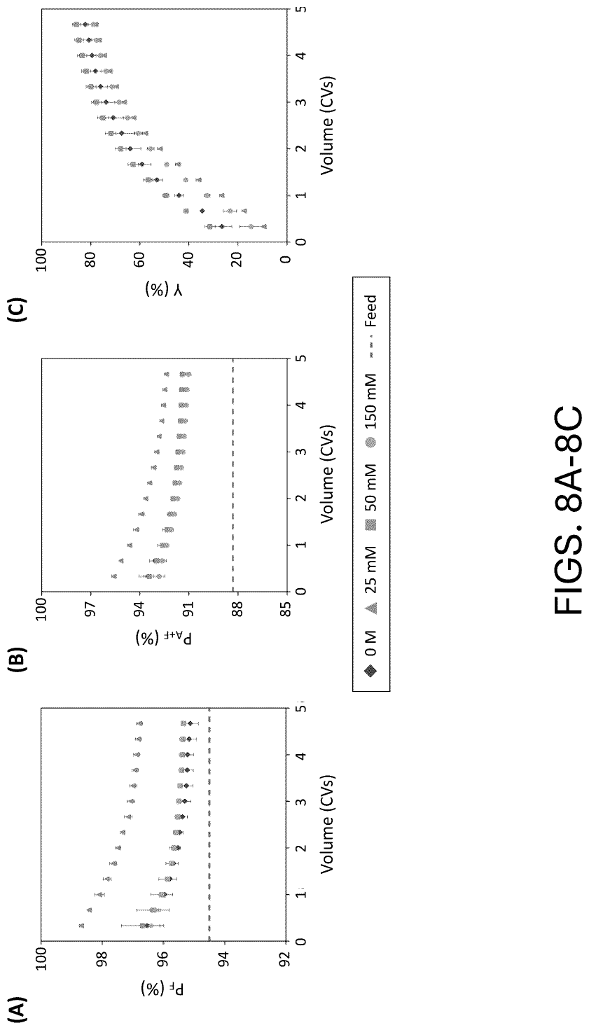

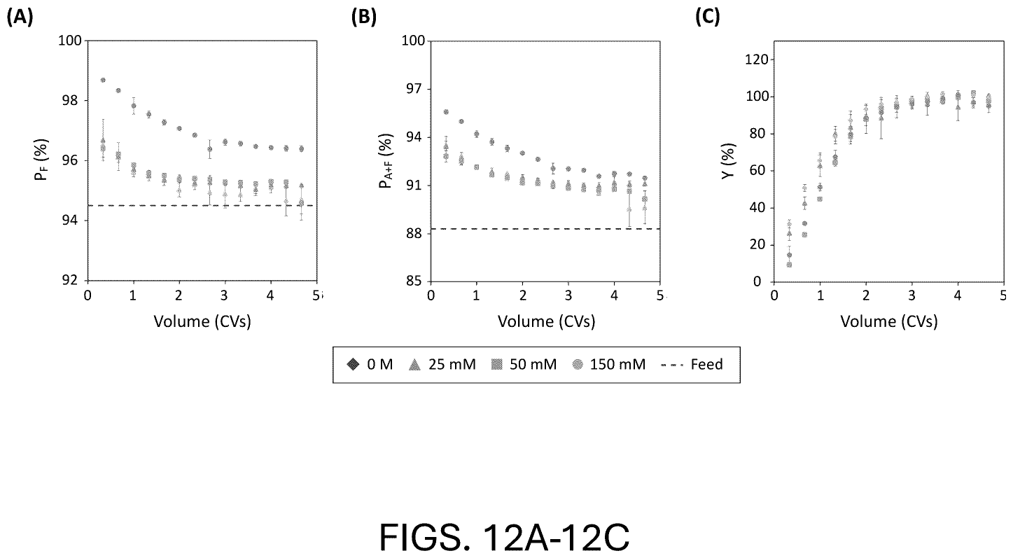

The addition of chaotropic salts to the mobile phase can in fact partially unfold the antibody fragments and expose their hydrophobic residues, thus promoting their interaction with hydrophobic moieties of MM ligands. Accordingly, the study focused on (i) DTS-Chromatorex resin, since APTES and TMTMAC do not comprise any hydrophobic moiety; and (ii) NaCl as a mild chaotropic salt, whose concentration was limited to the range of 25-150 mM to avoid disrupting the electrostatic component of fragment binding. The temporal profiles of cumulative purity and yield of the antibody monomer are in FIG. 8, and selected values corresponding to different load volumes are reported in Table 6; the fractional values of purity and yield are in FIG. 12.

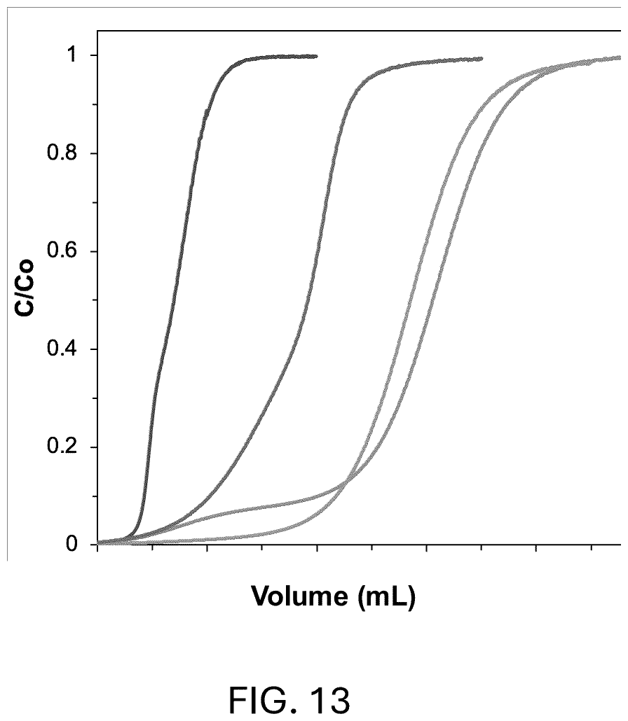

As anticipated, the addition of a small amount of salt (25 mM NaCl) to the mobile phase afforded a significant increase in product purity and widened the window of column load within which high IgG purity is obtained without affecting the temporal profile of yield. Notably, the monomeric product purity and yield rose to above 97% and 80%, respectively; the breakthrough curves of monomeric IgG and antibody fragments, reported in FIG. 13, confirmed the selective and capacitive capture of fragments, and thus the effectiveness of SEMM-silica resin for IgG polishing in flow-through mode.

TABLE 5
Values of cumulative yield (Yₙ) and purity of monomeric IgG calculated with respect to the capture
of IgG fragments only (P_{F, n}) obtained in the n^{th} fraction (i.e., value of load in column volumes,
CVs) at different values of pH. The values are extracted from the temporal profiles in FIG. 7.

| | PH | | | | | | | |
|---|---|---|---|---|---|---|---|---|
| | pH 6 | | PH 7 | | pH 8 | | pH 9 | |
| Load (n) | Yₙ | P_{F, n} | Yₙ | P_{F, n} | Yₙ | P_{F, n} | Yₙ | P_{F, n} |

APTES-Chromatorex resin

| | | | | | | | | |
|---|---|---|---|---|---|---|---|---|
| 0.7 CVs | 26.4 ± 12.3% | 6.4 ± 0.3% | 14.41^{†}% | 98.31^{†}% | 9.0 ± 3.0% | 93.9 ± 0.2% | 20.3 ± 2.9% | 95.8 ± 0.6% |
| 1.7 CVs | 49.3 ± 16.5% | 96.2 ± 0.1% | 40.1 ± 9.3% | 97.3 ± 0.3% | 19.9 ± 5.1% | 94.2 ± 0.2% | 45.5 ± 11.0% | 95.7 ± 0.4% |
| 2.7 CVs | 65.1 ± 14.2% | 95.9 ± 0.2% | 56.6 ± 9.1% | 96.7 ± 0.2% | 36.2 ± 7.2% | 94.3 ± 0.1% | 61.7 ± 13.8% | 95.8 ± 0.3% |
| 3.7 CVs | 74.3 ± 11.9% | 95.6 ± 0.2% | 67.3 ± 8.4% | 95.9 ± 0.1% | 48.8 ± 7.8% | 94.5 ± 0.1% | 72.1 ± 14.4% | 95.8 ± 0.2% |
| 4.7 CVs | 79.7 ± 11.0% | 95.4 ± 0.2% | 74.5 ± 7.9% | 95.9 ± 0.1% | 57.6 ± 7.5% | 94.6 ± 0.1% | 78.8 ± 14.6% | 95.8 ± 0.2% |

TMTMAC-Chromatorex resin

| | | | | | | | | |
|---|---|---|---|---|---|---|---|---|
| 0.7 CVs | 38.0 ± 3.8% | 96.7 ± 0.8% | 24.3 ± 0.6% | 97.3 ± 0.2% | 8.7 ± 2.2% | 93.0 ± 0.2% | 16.1 ± 8.8% | 94.8 ± 0.2% |
| 1.7 CVs | 56.7 ± 8.0% | 96.3 ± 0.4% | 43.1 ± 1.0% | 96.2 ± 0.3% | 18.1 ± 1.3% | 93.0 ± 0.2% | 31.7 ± 3.5% | 95.3 ± 0.1% |
| 2.7 CVs | 71.1 ± 6.3% | 95.9 ± 0.3% | 56.1 ± 2.6% | 95.7 ± 0.3% | 30.0 ± 0.6% | 93.6 ± 0.3% | 47.8 ± 2.4% | 95.5 ± 0.2% |
| 3.7 CVs | 78.1 ± 6.3% | 95.8 ± 0.1% | 64.3 ± 3.2% | 95.4 ± 0.4% | 40.2 ± 2.3% | 93.9 ± 0.2% | 58.0 ± 1.1% | 95.5 ± 0.2% |
| 4.7 CVs | 88.51^{†}% | 95.7 ± 0.1% | 70.5 ± 3.0% | 95.2 ± 0.4% | 48.2 ± 3.5% | 94.0 ± 0.1% | 65.7 ± 1.2% | 95.5 ± 0.2% |

DTS-Chromatorex resin

| | | | | | | | | |
|---|---|---|---|---|---|---|---|---|
| 0.7 CVs | 34.0 ± 4.7% | 96.4 ± 0.4% | 34.5 ± 0.3% | 96.3 ± 0.3 | 30.7 ± 3.4% | 95.4 ± 0.2% | 36.7 ± 4.6% | 95.4 ± 0.1% |
| 1.7 CVs | 60.2 ± 5.7% | 95.7 ± 0.2% | 59.1 ± 3.7% | 95.7 ± 0.1 | 52.8 ± 0.2% | 94.9 ± 0.3% | 59.0 ± 2.4% | 95.2 ± 0.1% |
| 2.7 CVs | 72.7 ± 6.6% | 95.5 ± 0.1% | 70.8 ± 5.4% | 95.4 ± 0.2 | 66.6 ± 2.9% | 94.7 ± 0.3% | 69.4 ± 0.4% | 95.1 ± 0.2% |
| 3.7 CVs | 80.8 ± 5.4% | 95.4 ± 0.1% | 78.1 ± 5.0% | 95.2 ± 0.2 | 74.4 ± 4.0% | 94.8 ± 0.2% | 75.7 ± 0.4% | 95.1 ± 0.2% |
| 4.7 CVs | 85.6 ± 5.1% | 95.3 ± 0.1% | 82.2 ± 4.7% | 95.1 ± 0.3% | 79.3 ± 4.5% | 94.8 ± 0.2% | 80.4 ± 0.6% | 95.0 ± 0.1% |

TABLE 6
Values of cumulative purity of fragments only (P_{F, n}) and recovery (Yₙ), of monomeric IgG calculated
with respect to the capture of IgG fragments, obtained with DTS-Chromatorex resin at different
values of mobile phase ionic strength. The values are extracted from the profiles in FIG. 8.

| | Salinity | | | | | | | |
|---|---|---|---|---|---|---|---|---|
| | 0M | | 25 mM | | 50 mM | | 150 mM | |
| Load (n) | Yₙ | P_{F, n} | Yₙ | P_{F, n} | Yₙ | P_{F, n} | Yₙ | P_{F, n} |
| 0.7 CVs | 35.5 ± 0.3% | 96.3 ± 0.2% | 17.4 ± 0.9% | 98.4 ± 0.1% | 41.1 ± 0.1% | 96.3 ± 0.5% | 23.2 ± 2.7% | 96.3 ± 0.1% |
| 1.7 CVs | 59.1 ± 3.7% | 95.7 ± 0.1% | 44.5 ± 1.1% | 97.6 ± 0.1% | 62.7 ± 2.1% | 95.7 ± 0.2% | 49.0 ± 0.8% | 95.7 ± 0.1% |
| 2.7 CVs | 70.8 ± 5% | 95.4 ± 0.2% | 62.4 ± 0.8% | 97.2 ± 0.1% | 75.0 ± 2.2% | 95.5 ± 0.1% | 65.0 ± 1.8% | 95.6 ± 0.1% |
| 3.7 CVs | 78.1 ± 5.4% | 95.2 ± 0.2% | 72.1 ± 0.7% | 96.9 ± 0.1% | 81.8 ± 1.9% | 95.4 ± 0.1% | 73.8 ± 1.9% | 95.4 ± 0.1% |
| 4.7 CVs | 82.2 ± 4.7% | 95.1 ± 0.3% | 78.1 ± 0.6% | 96.8 ± 0.1% | 85.8 ± 1.7% | 95.4 ± 0.1% | 78.9 ± 2.0% | 95.3 ± 0.1% |

TABLE 7
Transport and isotherm parameters used for each protein component in breakthrough
curve simulations for the 100 Å pore diameter resin.

| | mAb | Fab₂ | Fab/Fc | LMW |
|---|---|---|---|---|
| Dₐₓ [m²/s] | 3.04 × 10⁻⁹ | 3.04 × 10⁻⁹ | 3.04 × 10⁻⁹ | 3.04 × 10⁻⁹ |
| k_{film} [m/s] | 6.08 × 10⁻⁶ | 6.70 × 10⁻⁶ | 7.92 × 10⁻⁶ | 9.36 × 10⁻⁶ |
| Dₚ [m²/s] | 1.74 × 10⁻¹⁶ | 8.32 × 10⁻¹⁵ | 1.40 × 10⁻¹³ | 6.58 × 10⁻¹³ |
| k_{L} [m³/mol] | 1.13 × 10⁶ | 0.75 × 10⁶ | 0.375 × 10⁶ | 0.188 × 10⁶ |
| qₘₐₓ [mol/m³] | 0.833 | 1.25 | 2.5 | 5 |

TABLE 8
Transport and isotherm parameters used for each protein component in breakthrough
curve simulations for the 200 Å pore diameter resin.

| | mAb | Fab₂ | Fab/Fc | LMW |
|---|---|---|---|---|
| Dₐₓ [m²/s] | 3.04 × 10⁻⁹ | 3.04 × 10⁻⁹ | 3.04 × 10⁻⁹ | 3.04 × 10⁻⁹ |
| k_{film} [m/s] | 6.08 × 10⁻⁶ | 6.70 × 10⁻⁶ | 7.92 × 10⁻⁶ | 9.36 × 10⁻⁶ |
| Dₚ [m²/s] | 4.14 × 10⁻¹³ | 7.57 × 10⁻¹³ | 1.71 × 10⁻¹² | 3.24 × 10⁻¹² |
| k_{L} [m³/mol] | 1.13 × 10⁶ | 0.75 × 10⁶ | 0.375 × 10⁶ | 0.188 × 10⁶ |
| qₘₐₓ [mol/m³] | 1.39 | 2.08 | 4.17 | 8.33 |

TABLE 9
Transport and isotherm parameters used for each protein component in breakthrough
curve simulations for the 300 Å pore diameter resin.

| | mAb | Fab₂ | Fab/Fc | LMW |
|---|---|---|---|---|
| Dₐₓ [m²/s] | 3.04 × 10⁻⁹ | 3.04 × 10⁻⁹ | 3.04 × 10⁻⁹ | 3.04 × 10⁻⁹ |
| k_{film} [m/s] | 6.08 × 10⁻⁶ | 6.70 × 10⁻⁶ | 7.92 × 10⁻⁶ | 9.36 × 10⁻⁶ |
| Dₚ [m²/s] | 1.17 × 10⁻¹² | 1.73 × 10⁻¹² | 3.04 × 10⁻¹² | 4.91 × 10⁻¹² |
| k_{L} [m³/mol] | 1.13 × 10⁶ | 0.75 × 10⁶ | 0.375 × 10⁶ | 0.188 × 10⁶ |
| qₘₐₓ [mol/m³] | 1.94 | 2.92 | 5.83 | 11.7 |

### 5. Materials and Methods

Materials. A solution of dimethyloctadecyl[3-(trimethoxysilyl)propyl]ammonium chloride (DTS) at 42% w/w in methanol, methanol, sodium phosphate monobasic dihydrate, sodium phosphate dibasic dihydrate, potassium phosphate monobasic, potassium chloride, sodium azide, tris(hydroxymethyl)aminomethane (TRIS), sulfobenzoic acid (SBA), cysteic acid (CA), and Fmoc-protected trimethyl-lysine (Fmoc-Lys(Me)3-OH) and naphthalyl-alanine (Fmoc-1-Na1-OH) were purchased from MilliporeSigma (Burlington, MA). N,N′-dimethylformamide (DMF), dichloromethane (DCM), Pierce BCA kits, hydrochloric acid, sodium chloride, and a solution of N-[3-(Trimethoxysilyl)propyl]-N,N,N-trimethylammonium chloride (TMTAC) at 50% w/w in methanol were purchased from ThermoFisher Scientific (Waltham, MA). Human polyclonal IgG, and its F(ab)2, Fab, Fc fragments were purchased from Athens Research and Technology (Athens, GA). Azabenzotriazole tetramethyl uronium hexafluorophosphate (HATU) and diisopropylethylamine (DIPEA), piperidine were sourced from ChemImpex International (Wood Dale, IL, USA). The Chromatorex silica resins (primary amine density: 0.2 mmol per gram of resin; spherical particle size: 65 μm; pore diameter: 100 Å (polydispersity index of pore diameter, PDI˜0.04), 200 Å (PDI˜0.04), and 300 Å (PDI˜0.05); note: the profile pore size distribution is proprietary information of Fuji Silysia) functionalized with (3-aminopropyl)triethoxysilane (APTES) were donated by Fuji Silysia Chemical LTD (Kasugai, Japan). The BioResolve SEC mAb column (200 Å, 2.5 mm, 7.8×300 mm) was from Waters Corporation (Milford, MA). The Microbore PEEK columns 2.1 mm ID×100 mm, 2.1 mm ID×30 mm, 5 mm ID×50 mm, 5 mm ID×25 mm, and 10 mm ID×5 mm were purchased from VICI Precision Sampling (Baton Rouge, LA).

Production of SEMM-silica resins. An aliquot of 2 g of Chromatorex silica resin was initially incubated overnight at 60° C. The dry resin was then suspended in methanol and incubated under mild stirring with 2.8 mL of either a 42% w/w solution of DTS in methanol or a 50% w/w solution of TMTMAC in methanol overnight at room temperature in the dark. Alternatively, the dry resin was incubated with a DMF solution of 3 equivalents compared to the primary amine density (3 eq) of HATU (456 mg), DIPEA (209 L), and either SBA (242 mg) or Fmoc-Lys(Me)3-OH (536 mg) or Fmoc-1-Na1-OH (525 mg) for 30 min at 75° C.; the reactions were performed two times with intermediate washing with 6 mL of DMF (3×). The SBA-Chromatorex resin was then suspended in methanol and incubated under mild stirring with 2.8 mL of a 50% w/w solution of TMTMAC in methanol overnight at room temperature in the dark. The Fmoc-Lys(Me)3-Chromatorex and Fmoc-1-Na1-Chromatorex resins were then incubated with 6 mL of 20% v/v piperidine in DMF (2×) under mild stirring for 2 hours at room temperature to remove the Fmoc protecting group, and thoroughly washed with 6 mL of DMF (3×). The NH2-Lys(Me)3-Chromatorex resin was incubated with a DMF solution of 3 eq. of HATU (456 mg), DIPEA (209 L), and SBA (242 mg) for 30 min at 75° C.; the NH2-1-Na1-Chromatorex resin was incubated with a DMF solution of 3 eq. of HATU (456 mg), DIPEA (209 L), and CA (203 mg) for 30 min at 75° C. Following functionalization, the resulting DTS-Chromatorex, TMTMAC-Chromatorex, SBA-Chromatorex, SBA-Lys(Me)3-Chromatorex, CA-1-Na1-Chromatorex, and APTES-Chromatorex were washed with 6 mL of methanol (3×), 6 mL of DMF (3×), and 6 mL of DCM (3×), dried under a nitrogen flow, and stored dry at room temperature. The values of ligand density of the resultant SEMM-silica adsorbents were determined via elemental analysis and are reported in Table 10.

TABLE 10
Values of ligand density measured via elemental
analysis of SEMM-silica resins.

| | Pore Size | Ligand density |
|---|---|---|
| Ligand | (Å) | (mmol/g) |
| APTES | 300 | 0.17 |
| | 200 | 0.21 |
| | 100 | 0.24 |
| TMTMAC | 300 | 0.11 |
| | 200 | 0.12 |
| | 100 | 0.14 |
| DTS | 300 | 0.13 |
| | 200 | 0.16 |
| | 100 | 0.19 |
| SBA | 100 | 0.15 |
| SBA-TMTMAC | | 0.14 |
| SBA-Lys(Me)₃ | | 0.11 |
| CA-1-Nal | | 0.12 |

Static binding isotherms of human polyclonal IgG, and its Fc, Fab, and F(ab)2 fragments on SEMM-Chromatorex resins with different pore diameter. Static binding studies were performed on DTS-Chromatorex, TMTMAC-Chromatorex, and APTES-Chromatorex resins (pore diameter: 100 Å, 200 Å, and 300 Å) using feedstock solutions of human IgG (1-40 g/L), Fc fragment (0.25-2 g/L), or Fab fragment (0.25-2 g/L) in 50 mM Tris HCl at pH 8.0. Briefly, L of silica resin (settled volume) were incubated with 200 L of feedstock protein solution for 2.5 hours under gentle agitation. The resin was then pelleted by centrifugation and the supernatant was analyzed to measure the residual protein concentration in solution at the equilibrium (C*). The mass of protein bound per volume of resin (q) was calculated via mass balance. The adsorption data were fit against a Langmuir isotherm (Equation 1) to determine the values of maximum binding capacity (Qmax) and dissociation constant (KD).

$$q = \frac{{Q}_{max} {C}^{*}}{{K}_{D} + {C}^{*}} \qquad Equation 1$$

Evaluation of IgG fragment removal by SEMM-Chromatorex silica resins. The DTS-Chromatorex, TMTMAC-Chromatorex, SBA-Chromatorex, SBA/TMTMAC-Chromatorex, SBA-Lys(Me)3-Chromatorex, and CA-1-Na1-Chromatorex, and APTES-Chromatorex resins (pore diameter: 100 Å) were packed in a 0.1 mL Microbore PEEK column (2.1 mm ID×30 mm) and mounted on a Waters Alliance 2695 HPLC System equipped with a 2487 Dual Absorbance Detector. The resins were equilibrated in 5 column volumes (CVs) of 50 mM Tris HCl at pH 8.0 at the linear velocity of 173 cm/h. Feedstock solutions of human polyclonal IgG at 15 g/L, Fc fragment at 2.5 g/L, or Fab fragment at 2.5 g/L were prepared in 50 mM Tris HCl buffer at pH 8.0. A volume of 0.1 mL of feedstock solution was loaded on the column at 173 cm/h and the effluent was monitored via UV spectroscopy at 280 nm for 20 CVs. The capture of the kth protein (i.e., IgG, Fe, or Fab) by the ith (i.e., DTS-, TMTMAC-, SBA-, SBA/TMTMAC-, SBA-Lys(Me)3-, and CA-1-Na1-, or APTES-) ligand (Ck,i) was determined using Equation 2:

$${C}_{k , i} ( \% ) = ( 1 - \frac{{A}_{k , i}}{{A}_{k}} ) \times 100 \% \qquad Equation 2$$

Wherein Ak,i is the area of the chromatogram generated by loading the kth protein on a column packed with the ith Chromatorex resin, while Ak is the area of the chromatogram generated by loading the kth protein on an empty column. The values of capture C_{k,i} were utilized to calculate a purification score for the ith resin (Pi, ranges from 0 to 1) using Equation 3:

$${P}_{i} = ( 100 \% - {C}_{IgG , i} ) \times {C}_{F c , i} \times {C}_{Fab , i} \qquad Equation 3$$

Capture of antibody fragments by SEMM-silica resin in flow-through mode. Seven binding buffers were prepared, namely 50 mM sodium phosphate buffer at pH 6 and 7, 50 mM sodium phosphate buffer and X mM NaCl (X: 25, 50, or 150) at pH 7, and 50 mM Tris HCl at pH 8 and 9. Model Protein A eluates was prepared as solutions of human monomeric IgG (5 g/L), IgG aggregate (0.35 g/L), Fab2 (0.25 g/L), Fc/Fab (0.28 g/L), and low molecular weight fragments (0.02 g/L) in each binding buffer. DTS-Chromatorex, TMTMAC-Chromatorex, and APTES-Chromatorex resins were packed in a 0.3 mL Microbore PEEK column (2.1 mm ID×30 mm) and equilibrated in 5 CVs of binding buffer. A volume of 2 mL of feedstock was loaded on the column at the linear velocity of either 200, 150, 120, 100, or 85.7 cm/h, corresponding to residence times of 3, 4, 5, 6, and 7 min; the effluent was continuously monitored via UV spectroscopy at 280 nm for 5 CVs. The flow-through fractions were analyzed via analytical size exclusion chromatography (SEC-HPLC) as described in Section 2.6 to determine the yield and purity of monomeric IgG in the effluent. Finally, measurements of dynamic binding capacity of monomeric IgG and IgG fragments were conducted by loading the 0.3 mL column packed with DTS-Chromatorex resin with 5 mL of solution of pure protein—namely IgG (5 mg/mL), Fab2 (0.25 g/L), Fc and Fab (0.5 g/L) in 50 mM sodium phosphate buffer added with 25 mM NaCl at pH 7—at the residence time of 6 min; the effluent was continuously monitored via UV spectroscopy at 280 nm.

Analytical size exclusion chromatography (SEC) for the quantification of monomeric antibody purity. The effluents collected from the SEMM-silica columns were analyzed for molecular weight distribution using a BioResolve SEC mAb column with PBS at pH 7.4 as mobile phase. A sample volume of 20 L was injected at the flowrate of 0.5 mL/min and the UV absorbance of the effluent was continuously monitored at 280 nm. The resulting chromatograms were divided into (i) “high molecular weight” segment containing antibody aggregates (HMW, MW>175 kDa, retention time: 0-10.0 min), (ii) “monomeric antibody” peak (IgG, 125 kDa<MW<175 kDa, retention time: 13.2-16.2 min), (iii) “Fab2” segment (75 kDa<MW<125 kDa, retention time: 15.2-15.7 min), (iv) “Fc and Fab” segment (Fc/Fab, 40 kDa<MW<75 kDa, retention time: 17.2-19.0 min), and (v) “low molecular weight fragments” segment (LMW, MW<40 kDa, retention time: 20-40 min). The corresponding peak areas AHMW, AIgG, AFab2, AFc/Fab, and ALMW were utilized to calculate the values of fractional yield (obtained in the jth fraction, γ_{i,j}) and cumulative yield (from the onset of loading until the nth fraction, Y_{i,n}), where i denotes the species (HMW, IgG, Fab2, Fc/Fab, and LMW) whereas j and f denote the jth and nth flow-through fractions (1<j<n); as well as the fractional purity (pⱼ) and the cumulative purity (Pₙ) of the monomeric antibody product using Equations 4-7:

$${y}_{i , j} ( \% ) = \frac{{A}_{i , j}}{{A}_{i , feedstock}} \times 100 \% \qquad Equation 4$$

$${y}_{i , n} ( \% ) = \frac{{\sum }_{j = 1}^{n} {A}_{i , j}}{n \times {A}_{i , feedstock}} \times 100 \% \qquad Equation 5$$

$${p}_{j , F} ( \% ) = \frac{{A}_{IgG , j}}{{A}_{IgG , j} + {A}_{Fab 2 , j} + {A}_{Fc / Fab , j} + {A}_{LMW , j}} \times 100 \% \qquad Equation 6 a$$

$${p}_{j , F + A} ( \% ) = \frac{{A}_{IgG , j}}{{A}_{HMW , j} {A}_{IgG , j} + {A}_{Fab 2 , j} + {A}_{Fc / Fab , j} + {A}_{LMW , j}} \times 100 \% \qquad Equation 6 b$$

$${p}_{n , F} ( \% ) = \frac{{\sum }_{j = 1}^{f} {A}_{IgG , j}}{{\sum }_{j = 1}^{f} {A}_{IgG , j} + {A}_{Fab 2 , j} + {A}_{Fc / Fab , j} + {A}_{LMW , j}} \times 100 \% \qquad Equation 7 a$$

$${p}_{n , F \& A} ( \% ) = \frac{{\sum }_{j = 1}^{n} {A}_{IgG , j}}{{\sum }_{j = 1}^{n} {A}_{HMW , j} + {A}_{IgG , j} + {A}_{Fab 2 , j} + {A}_{Fc / Fab , j} + {A}_{LMW , j}} \times 100 \% \qquad Equation 7 b$$

Note that the values of monomeric purity were calculated with respect to the removal of fragments only (P_{j,F} and P_{n,F}, Equations 6a and 7a) or both aggregates and the fragments (P_{j,F+A} and P_{n,F&A}, Equations 6b and 7b).

Breakthrough curve simulations. Three pore sizes (100, 200, and 300 Å diameter) were investigated with respect to their efficacy in removing antibody fragments using a mechanistic modeling-based approach. To model the transport of protein through the column, the general rate model described by Equations 8a and 8b, in concert with the multicomponent Langmuir isotherm (Equation 9) in its kinetic form were employed.

$$\frac{\partial {c}_{i}}{\partial t} = - {u}_{int} \frac{\partial {c}_{i}}{\partial x} + {D}_{ax , i} \frac{{\partial }^{2} {c}_{i}}{\partial {x}^{2}} - \frac{1 - {ε}_{e}}{{ε}_{e}} \frac{3}{{r}_{part}} {k}_{film , i} [ {c}_{i} - {c}_{p , i} ] \qquad Equation 8 a$$

$$\frac{\partial {c}_{p , i}}{\partial t} = {D}_{p , i} ( \frac{{\partial }^{2} {c}_{p , i}}{\partial {r}^{2}} + \frac{2}{r} \frac{\partial {c}_{p , i}}{\partial r} ) + {D}_{s , i} ( \frac{{\partial }^{2} {q}_{i}}{\partial {r}^{2}} + \frac{2}{r} \frac{\partial {q}_{i}}{\partial r} ) - \frac{1 - {ε}_{p}}{{ε}_{p}} \frac{\partial {q}_{i}}{\partial t} \qquad Equation 8 b$$

$$\frac{\partial {q}_{i}}{\partial t} = {k}_{L , i} {c}_{p , i} {q}_{max , i} ( 1 - {\sum }_{j = 0}^{{N}_{comp} - 1} \frac{{q}_{j}}{{q}_{max , j}} ) - {q}_{i} \qquad Equation 9$$

Representative Langmuir isotherm constants k_{L,i} and q_{max,i} were obtained from the data collated in Tables 1 and 2, and converted to units of solid resin volume using the total porosity, calculated using interstitial porosity Ee of 0.4 and intraparticle porosity E, of 0.6. Transport parameters for axial dispersion D_{ax,i} and film mass transfer rate k_{film,i} were estimated using the correlations from Rastegar and Gu (See, Rastegar, S. O. & Gu, T. J. Chromatogr. A 1490, 133-137 (2017)) and Wilson-Geankopolis (See, Wilson, E. J. & Geankoplis, Ind. Eng. Chem. Fundam. 5, 9-14 (1966)), respectively. Pore diffusivity D_{p,i} was estimated using the 7th order pore hindrance correlation from Higdon and Muldowney, described by Dechadilok and Deen (See, doi:10.1021/ie051387n), and the effective pore diffusivity relation. The pore hindrance correlation is based on the ratio of protein hydrodynamic radius to resin pore radius and was thus calculated from these quantities. Surface diffusivity D_{s,i} was set to zero as it was expected to be unimpactful under conditions of high protein affinity. The remaining model parameters were either obtained from the manufacturer or inferred from typical values preparative chromatographic resins. The isotherm and transport parameters used in these simulations are reported in Tables 7-9.

Simulations were performed using the CADET modeling software using a MATLAB-based front-end interface. These simulations consisted of a 15 CV protein loading phase followed by a 2 CV wash for a 0.5×2.5 cm column. The same protein mixture was used for all simulations and contained four components: mAb (150 kDa), Fab2 (100 kDa), Fab/Fc (50 kDa), and LMW species (25 kDa). Total protein concentration was set to 6 mg/mL and the mass fractions were 0.9, 0.03, 0.05, and 0.02, respectively. Simulations for each of the pore sizes were carried out at residence times r of 2, 6, and 10 minutes. The resulting breakthrough curves were compared to determine which pore size was most suitable for overall fragment removal with minimal mAb loss.

What is claimed is:

1. A method for purifying a target biologic comprising:
contacting a sample comprising the target biologic with a multifunctional chromatography medium comprising a solid substrate, wherein the solid substrate comprises a narrow pore diameter distribution, and one or more ligands functionalized to surfaces of the pores, wherein the one or more ligands are configured to bind or associate with at least one impurity in the sample; and
collecting the target biologic.

2. The method of claim 1, wherein the solid substrate comprises a plurality of beads or particles, or a monolith.

3. The method of claim 1 or 2, wherein the target biologic is one or more of a peptide, a polypeptide, or a protein; an oligonucleotide or a polynucleotide; a virus or a virus-like particle; an exosome or an extracellular vesicle; a cell or cell organelle; or a small molecule.

4. The method of any one of claims 1-3, wherein the target biologic is an antibody.

5. The method of any one of claims 1-4, wherein the at least one impurity comprises a process-related impurity, a product-related impurity, or a combination thereof.

6. The method of any one of claims 1-5, wherein the at least one impurity comprises a fragment of the target biologic, and the method comprises a step of separating the target biologic from the fragment of the target biologic.

7. The method of any of claims 1-6, wherein the ligand has a high binding strength for the at least one impurity.

8. The method of any of claims 1-7, wherein the ligand binds or associates to the at least one impurity with a dissociation constant (K_{d}) of about 10⁻³ M to about 10⁻⁹ M.

9. The method of claim 7 or 8, wherein the at least one impurity is part of an aggregate comprising the target biologic and the at least one impurity is partitioned from the aggregate.

10. The method of any one of claims 1-9, wherein the pore diameter distribution is configured to exclude the target biologic.

11. The method of any one of claims 1-10, wherein the pore diameter distribution has an average pore diameter of about 50 Å to about 1000 Å.

12. The method of claim 11, wherein the average pore diameter is about 100 Å to about 500 Å.

13. The method of any one of claims 1-12, wherein the pore diameter distribution is monodisperse.

14. The method of any one of claims 1-13, wherein the plurality of beads or particles comprise silica, polyacrylamide, polymethacrylate, polystyrene, polyvinylether, or combinations thereof.

15. The method of any one of claims 1-14, wherein the plurality of beads or particles are silica beads or particles.

16. The method of any one of claims 1-15, wherein the plurality of beads or particles have a particle size ranging between 20 to 150 μm.

17. The method of any one of claims 1-16, wherein each of the one or more ligands are selected from: a small molecule, a biomolecule, a metal, or a combination thereof.

18. The method of claim 17, wherein the biomolecule comprises a nucleic acid, an oligonucleotide, an amino acid, a peptide, a monosaccharide, an oligosaccharide, a lipid, a steroid, or a conjugate thereof.

19. The method of any one of claims 1-18, wherein each of the one or more ligands comprise an affinity ligand, an ion exchange ligand, a hydrophobic ligand, a hydrophilic ligand, a polar ligand, a thiophilic ligand, or a combination thereof.

20. The method of claim 19, wherein the ion exchange ligand is negatively charged.

21. The method of claim 20, wherein the ion exchange ligand comprises a sulfate group, a phosphate group, a carboxylate group, or a combination thereof.

22. The method of any one of claims 19-21, wherein the ion exchange ligand is positively charged.

23. The method of claim 22, wherein the ion exchange ligand comprises a primary, secondary, tertiary, or quaternary aminoalkyl group, an ammonium group, or an oligomer thereof or a polymer thereof; an imidazole group; a pyridinyl group; a guanidyl group; or a combination thereof.

24. The method of any one of claims 19-23, wherein the hydrophobic ligand comprises alkyl or aryl groups.

25. The method of any one of claims 19-24, wherein the affinity ligand is a biological affinity ligand, an immunoaffinity ligand, or a metal affinity ligand.

26. The method of any one of claims 1-25, wherein the chromatography medium is suspended in solution or packed in a column.

27. The method of any one of claims 1-26, wherein the method is performed under static binding conditions.

28. The method of claim 27, wherein the method comprises collecting the target biologic in the unbound fraction, wash fraction, or a combination thereof.

29. The method of any one of claims 1-28, wherein the method is performed under dynamic binding conditions.

30. The method of claim 29, wherein the method comprises collecting the target biologic in the flow through, column wash, or a combination thereof.

31. The method of any one of claim 1-30, wherein the method further comprises preparing the sample comprising the target biologic.

32. The method of claim 31, wherein preparing the sample comprising the target biologic comprises one or more chromatography steps.

33. The method of claim 31 or 32, wherein preparing the sample comprises carrying out affinity chromatography and collecting an eluate comprising the target biologic.

34. The method of any of claims 1-33, further comprising one or more additional chromatography steps following collecting the target biologic.

35. The method of any of claims 1-34, further comprising selecting the multifunctional chromatography medium.
